(19) United States
(12) Patent Application Publication
(10) Pub. No.: US 2025/0389728 A1
(43) Pub. Date: Dec. 25, 2025

(54) NOVEL CLASS OF TRIOXANE-BASED MS-CLEAVABLE CROSS-LINKERS TO ADVANCE PROTEIN-PROTEIN INTERACTION STUDIES USING CROSS-LINKING MASS SPECTROMETRY

(71) Applicant: The Regents of the University of California, Oakland, CA (US)

(72) Inventors: Lan Huang, Irvine, CA (US); Scott D. Rychnovsky, Irvine, CA (US); Eric J. Novitsky, Irvine, CA (US); Clinton Yu, Irvine, CA (US)

(21) Appl. No.: 19/232,844

(22) Filed: Jun. 9, 2025

Related U.S. Application Data
(60) Provisional application No. 63/663,067, filed on Jun. 21, 2024.

(51) Int. Cl. G01N 33/68

(52) U.S. Cl. CPC G01N 33/6848; G01N 33/6845; G01N 2560/00

(57) ABSTRACT

The disclosure provides for mass spectrometry (MS)-cleavable trioxane-based cross-linkers, and uses thereof, including for protein-protein interaction studies using cross-linking mass spectrometry.

### CROSS REFERENCE TO RELATED APPLICATIONS

This application claims priority under 35 U.S.C. § 119 from Provisional Application Ser. No. 63/663,067, filed Jun. 21, 2024 the disclosure of which is incorporated herein by reference.

### STATEMENT OF GOVERNMENT SUPPORT

This invention was made with Government support under Grant Nos. R01GM074830 and R35GM145249, awarded by the National Institutes of Health. The Government has certain rights in the invention.

### INCORPORATION BY REFERENCE OF SEQUENCE LISTING

Accompanying this filing is a Sequence Listing entitled, “00058-083001.xml” created on Jun. 9, 2025 and having 13,379 bytes of data, machine formatted on IBM-PC, MS-Windows operating system. The sequence listing is hereby incorporated by reference in its entirety for all purposes.

### TECHNICAL FIELD

The disclosure provides for mass spectrometry (MS)-cleavable trioxane-based cross-linkers, and uses thereof, including for protein-protein interaction studies using cross-linking mass spectrometry.

### BACKGROUND

Protein-protein interactions (PPIs) are essential for the assembly of protein complexes, the active molecular modules for controlling cellular functionality and modulating physiological states. In recent years, cross-linking mass spectrometry (XL-MS) has been proven effective for studying PPIs and elucidating architectures of protein complexes in vitro and in vivo at the systems-level. Compared to other PPI methods, XL-MS enables the capture of endogenous PPIs without cell engineering. Identification of cross-linked peptides concurrently reveals PPI identities and interaction contacts at specific residues, which provide distance constraints defined by a given cross-linker to help refine existing structures and elucidate structures of protein complexes through computational modeling. However, the heterogeneity and low abundance of cross-linked fragments remains a hindrance for analysis of higher-order cross-links. Therefore, it would be advantageous to implement an MS-cleavable bond that is weaker than peptide bonds in the design of multifunctional cross-linkers to minimize the number of fragment ions for subsequent MS³ analysis.

### SUMMARY

In recent years, chemical cross-linking coupled with mass spectrometry has provided a myriad of detailed insights into protein interactions and structures. By covalently binding pairs of proximal residues, cross-linking reagents provide distance constraints that inform on protein conformations and interaction interfaces. However, proteins often participate in various functional assemblies, and the coexistence of diverse multi-protein complexes can impede the proper assignment of binary cross-link information. To expand observations of protein connectivity and improve restraint information for structural modeling, the disclosure provides for the design and development of a novel trioxane-based MS-cleavable homotrifunctional cross-linker that can simultaneously target three proximal lysine residues.

In particular, the disclosure provides for the design, synthesis, and characterization of novel trioxane-based MS-cleavable, membrane-permeable homotrifuctional cross-linkers to dissect multimeric protein interactions. In the studies presented herein, an exemplary trioxane-based MS-cleavable cross-linker of the disclosure, TSTO (tris-succinimidyl trioxane), enabled simultaneous cross-linking of up to three proteins, allowing for more in-depth PPI analysis and providing additional restraints to advance structural analysis of protein assemblies. The studies demonstrated that all types of TSTO cross-linked peptides display unique and predictable CID-induced fragmentation and can be unambiguously identified using LC MSⁿ analysis. The trioxane-based MS-cleavable cross-linkers of the disclosure ability to concurrently release all three cross-link arms and leave an identical remnant on each cross-linked residue establishes it a brand-new class of MS-cleavable reagent. Additionally, this distinctive feature minimizes the number of theoretical MS fragments corresponding to each peptide constituent, simplifying ion selection for subsequent MS³ analysis.

In a particular embodiment, the disclosure provides a trioxane-based mass spectrometry (MS)-cleavable cross-linker comprising: a central trioxane group, which may be isotopically enriched with heavier isotopes selected from C¹³, and O¹⁸; two or more MS-cleavable bonds; two or more reactive cross-linking groups that can react with amino acids of peptides and/or proteins; optionally, linker arms that connect the reactive cross-linking groups with the central trioxane group, wherein the linker arms may be isotopically enriched with heavier isotopes selected from H², C¹³, O¹⁸ and N¹⁵; wherein the trioxane-based MS-cleavable cross-linker is configured to form a dimeric or trimeric cross-links with amino acids of peptides and/or proteins. In another embodiment, the two or more MS-cleavable bonds can be cleaved using collision-induced dissociation. In yet another embodiment, the two or more reactive cross-linking groups are located equal distant to the central trioxane group. In a further embodiment, the two or more reactive cross-linking groups are selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative. In yet a further embodiment, the central trioxane group comprises one or more of C¹³ and/or O¹⁸ atoms. In a certain embodiment, the trioxane-based MS-cleavable cross-linker comprises 2 or more linker arms that connect the reactive cross-linking groups with the central trioxane group, wherein the linker arms may be isotopically enriched with heavier isotopes selected from H², C¹³, O¹⁸ and N¹⁵. In another embodiment, one or more of the linker arms comprise one or more of H², C¹³, O¹⁸ and/or N¹⁵ atoms. In yet another embodiment, the trioxane-based mass spectrometry (MS)-cleavable cross-linker comprises 2 linker arms that connect the reactive cross-linking groups with the central trioxane group and comprise a linker arm that connects an enrichable handle or a fluorophore to the central trioxane group. In a further embodiment, the enrichable handle is selected from a click chemistry linker, a phosphate, a fluorophore, biotin, an azide, an alkyne, and a phosphonic acid. In yet a further embodiment, the trioxane-based MS-cleavable cross-linker comprises 2 linker arms that connect the reactive cross-linking groups with the central trioxane group and comprise a linker arm that connects an enrichable handle or a fluorophore to the central trioxane group. In another embodiment, the trioxane-based MS-cleavable cross-linker comprises 3 linker arms that connect three reactive cross-linking groups with the central trioxane group. In yet another embodiment, the three reactive cross-linking groups have different structures, or have different atomic masses. In a further embodiment, two of the reactive cross-linking groups have the same structure, and one of the reactive cross-linking groups has a different structure or has a different atomic mass. In yet a further embodiment, all three of the reactive cross-linking groups have the same structure. In another embodiment, one of the reactive cross-linking groups comprises one or more of H², C¹³, O¹⁸ and/or N¹⁵ atoms. In another embodiment, two of the reactive cross-linking groups comprises one or more of H², C¹³, O¹⁸ and/or N¹⁵ atoms. In yet another embodiment, the trioxane-based mass spectrometry (MS)-cleavable cross-linker has the structure of:

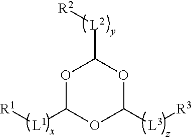

wherein, L¹, L², and L³ are linker arms each individually selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide,

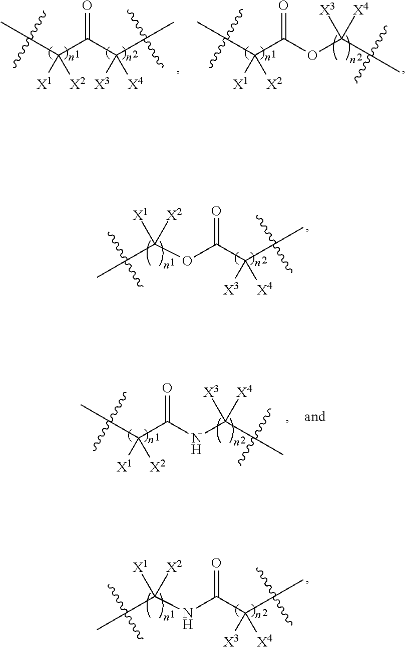

wherein L¹, L², and L³ may be isotopically enriched with heavier isotopes selected from H², C¹³, O¹⁸ and N¹⁵; R¹-R³ are individually selected from a reactive cross-linking group that can react with amino acids of peptides and/or proteins, an enrichable handle, and a fluorophore, wherein at least two of R¹ to R³ are reactive cross-linking groups; X¹-X⁴ are each individually selected from H, (C₁-C₆)alkyl, (C₁-C₆)alkenyl, (C₁-C₆)alkynyl, cyano, azide, hydroxyl, aldehyde, carboxyl, halo, amide, and amine, wherein each of the foregoing groups may be isotopically enriched with heavier isotopes selected from H², C¹³, O¹⁸ and/or N¹⁵; x, y, and z are integers selected from 0 and 1; and n¹ and n² are integers selected from 0, 1, 2, 3, 4, 5 and 6, wherein the trioxane-based MS-cleavable cross-linker is configured to form a dimeric or trimeric cross-links with amino acids of peptides and/or proteins. In yet a further embodiment, L¹, L², and L³ are individually selected from optionally substituted (C₁-C₆)alkyl,

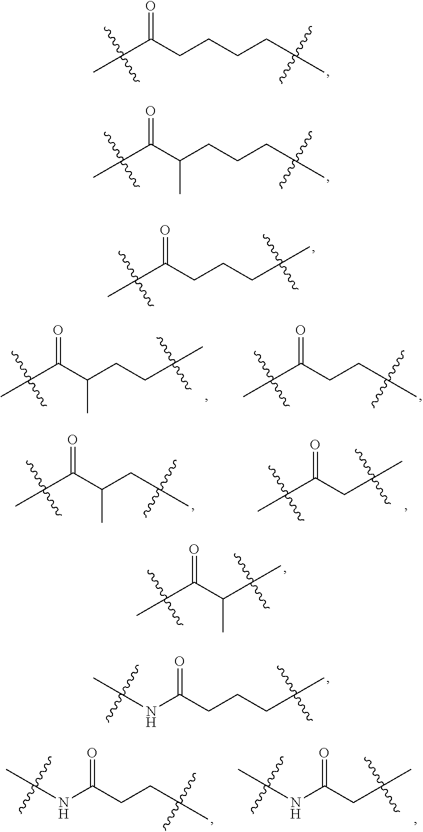

wherein each of the foregoing groups may be isotopically enriched with heavier isotopes selected from H², C¹³, O¹⁸ and N¹⁵. In another embodiment, L¹ to L³ are

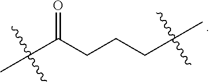 [SMILES: *CCCC(*)=O]

In yet another embodiment, the reactive cross-linking group is selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative. In a further embodiment, the reactive cross-linking group that can react with amino acids of peptides and/or proteins is selected from

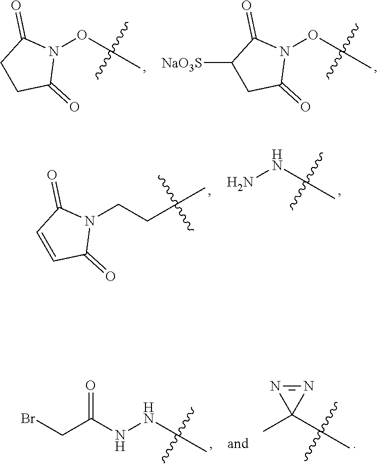

In a further embodiment, the enrichable handle is selected from a click chemistry linker, a phosphate, a fluorophore, biotin, an azide, an alkyne, and a phosphonic acid. In yet a further embodiment, R¹, R², and R³ are reactive cross-linking groups, and wherein: (i) at least two of R¹, R², and R³ have same structure, or (ii) R¹, R², and R³ have same structure, or (iii) R¹, R², and R³ have different structures. In yet a further embodiment, trioxane-based MS-cleavable cross-linker has the structure of:

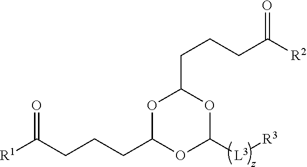

wherein, L³ is a linker arm selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide,

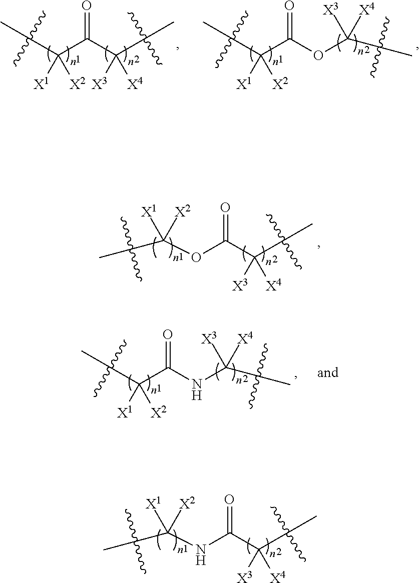

wherein L³ may be isotopically enriched with heavier isotopes selected from H², C¹³, O¹⁸ and N¹⁵; R¹ and R² are

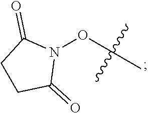 [SMILES: *ON1C(=O)CCC1=O]

R³ is selected from an enrichable handle, a fluorophore,

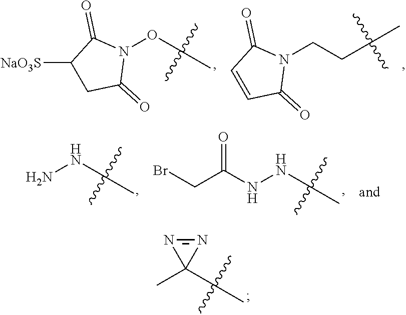

z is an integer selected from 0 and 1; and n¹ and n² are integers selected from 0, 1, 2, 3, 4, 5 and 6. In a certain embodiment, the MS-cleavable trioxane-based cross-linker has the structure of:

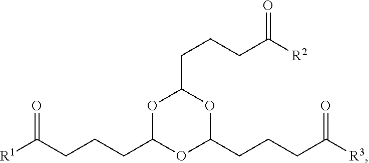

wherein, R¹-R³ are each individually selected from

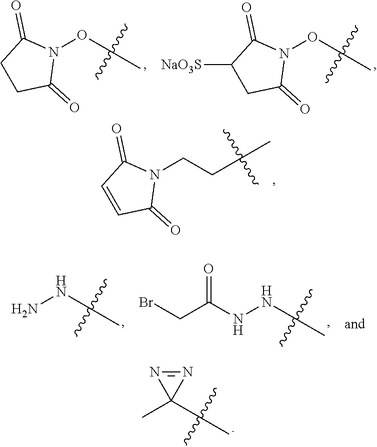

In a further embodiment, the trioxane-based MS-cleavable cross-linker is tris-succinimidyl trioxane (TSTO) having the structure of:

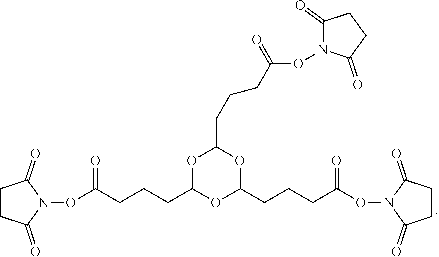 [SMILES: O=C(CCCC1OC(CCCC(=O)ON2C(=O)CCC2=O)OC(CCCC(=O)ON2C(=O)CCC2=O)O1)ON1C(=O)CCC1=O]

In a particular embodiment, the disclosure also provides a method for mapping intra-protein interactions in a protein, inter-protein interactions in a protein complex, or any combination thereof, the method comprising: contacting the protein and/or the protein complex comprising a plurality of cysteine moieties with the trioxane-based MS-cleavable cross-linker of any one of aspects 1 to 26 to form a cross-linked product; digesting the cross-linked product to form a plurality of fragments, wherein a portion of the plurality of fragments comprises cross-linked peptide fragments; and identifying and analyzing cross-linked peptide fragments using tandem mass spectrometry (MSⁿ) to map intra-protein interactions in the protein and/or inter-protein interactions in the protein complex. In another embodiment, the trioxane-based MS-cleavable cross-linker is used at a molar ratio of 1:10 to 10:1 to the protein and/or the protein complex. In yet another embodiment, the trioxane-based MS-cleavable cross-linker is used at a molar ratio of 1:5 to 5:1 to the protein and/or the protein complex. In a further embodiment, the cross-linked product was digested with one or more proteases. In yet a further embodiment, the one or more proteases are serine proteases. In another embodiment, a data-dependent MS³ acquisition method is used for identifying and analyzing the cross-linked peptide fragments. In yet another embodiment, the results of the data-dependent MS³ acquisition method is used with a computer program that preforms predictions of protein structure. In a further embodiment, the computer program is an artificial intelligence-based program. In yet a further embodiment, the computer program is a AlphaFold based program.

In a certain embodiment, the disclosure further provides a method for mapping global protein-protein interactions (PPIs) from a sample comprising a plurality of proteins; contacting the sample comprising a plurality of proteins with the trioxane-based MS-cleavable cross-linker of any one of aspects 1 to 26 to form crosslinked proteins; digesting the crosslinked proteins to form crosslinked protein fragments or peptides; isolating fractions that are enriched with cross-linked protein fragments or peptides in the sample; analyzing the fractions using tandem mass spectrometry (MSⁿ) and protein database searching to identify cross-linked protein fragments or peptides; and mapping the identified cross-linked protein fragments or peptides to generate a global structural map of PPIs. In another embodiment, the sample is a tissue sample or a cellular sample. In yet another embodiment, the cross-linked product was digested with one or more proteases. In a further embodiment, the one or more proteases are serine proteases. In yet a further embodiment, the fractions are isolated by using peptide size exclusion chromatography coupled with high pH reverse phase tip fractionation. In a certain embodiment, a data-dependent MS³ acquisition method is used for identifying the cross-linked protein fragments or peptides. In another embodiment, the identified cross-linked protein fragments or peptide are mapped using various databases that profile protein to protein interactions. In yet another embodiment, the databases or programs utilize artificial intelligence. In a further embodiment, the databases or programs is a AlphaFold based database or program.

In a certain embodiment, the disclosure provides a composition, a method or a kit as substantially described herein.

### DESCRIPTION OF DRAWINGS

FIG. 1A-B provides for TSTO design, synthesis, and expected cross-link formation and MSⁿ fragmentation. (A) TSTO synthesis. (B) TSTO cross-link products exhibit unique fragmentation during CID analysis. For tripeptide tri-links (Type I), MS² yields three fragment peptides with single AR-modified lysines. Dipeptide tri-links (Type II) produce two fragment peptides, one with a single AR-modified lysine and one with two. Finally, dipeptide bi-links (Type III) result in two fragment peptides, each with a single AR-modified lysine. MS³ analyses of these fragments enable unambiguous identification of TSTO tri- and bi-links.

FIG. 2A-C provides MSⁿ analyses of TSTO dead-end modified Ac-SR8. (A) MS² analysis of the dead-end modified Ac-SR8 [α_{DN}] (m/z 667.3163²⁺) yielded a dominant ion pair: α_{AR}/α_{AR*}, which has a mass difference of 18.02 Da. As the aldehyde moiety (AR) is prone to water loss, the dehydrated aldehyde moiety (AR*) was detected. (B) MS³ analysis of α_{AR} (m/z 551.2708²⁺) identified its sequence as Ac-SAK_{AR}AYEHR, in which K was modified by the aldehyde moiety (AR). (C) MS³ analysis of α_{AR*} (m/z 542.2656²⁺) identified its sequence as Ac-SAK_{AR*}AYEHR [SEQ ID NO:1], in which K was modified by the dehydrated aldehyde moiety (AR*).

FIG. 3A-C presents MS² fragmentation characteristics of TSTO inter-linked Ac-SR8. (A-B) MS² spectra of inter-linked Ac-SR8 homodimers [α-α]: (A) triply (m/z 773.7071³⁺) and (B) quadruply charged (m/z 580.5319⁴⁺). (C) MS² spectrum of the inter-linked Ac-SR8 homotrimer [α,α,α] (m/z 826.6508⁴⁺). Note: AR: aldehyde moiety; AR*: dehydrated aldehyde moiety.

FIG. 4A-F provides MSⁿ analyses of representative TSTO inter-linked peptides of BSA. (A) MS² spectrum of a tripeptide tri-link [α, β, γ] (m/z 795.7120⁶⁺) in which a series of dominant ions corresponding to α_{AR}, β_{AR}, and γ_{AR} fragments were detected. (B) MS³ sequencing of α_{AR}* (m/z 638.3130²⁺), β_{AR}* (m/z 773.874³⁺), and γ_{AR}* (m/z 947.934⁷⁺) enabled their identification as CASIQK_{AR*}FGER [SEQ ID NO:2], VTK_{AR*}CCTESLVNR [SEQ ID NO:3], and LAK_{AR*}EYEATLEECCAK [SEQ ID NO:4], respectively, signifying a tripeptide TSTO ti-link among BSA lysines K228, K374, and K498. (C) MS² spectrum of a dipeptide tri-link [α-β₂] (m/z 1054.7530⁴⁺), in which two sets of dominant ion species were observed: α_{AR}/α_{AR*}, and β_{2AR}/β_{AR_AR*}/β_{2AR*}. (D) MS³ analyses of α_{AR*} and β_{2AR*} identified their sequences as LAK_{AR*}EYEATLEECCAK [SEQ ID NO:4] and TPVSEK_{AR*}VTK_{AR*}CCTESLVNR [SEQ ID NO:5], signifying a dipeptide tri-link [BSA:K374-BSA:K495, K498]. (E) MS² spectrum of a dipeptide bi-link [α-β] (m/z 992.7017⁴⁺), in which two dominant ion pairs were detected: α_{AR}/α_{AR*}, and β_{AR}/β_{AR*}. (F) MS³ analyses of α_{AR*} (810.4316²⁺) and β_{AR*} (1099.4244²⁺) determined their sequences as LCVHEK_{AR*}TPVSEK [SEQ ID NO:6] and ETYGDMADCCEK_{AR*}QEPER [SEQ ID NO:7], identifying a bi-link between BSA:K117 and BSA:K489.

FIG. 5A-B presents 3-D distance mapping of TSTO cross-links of BSA. (A) 118 TSTO cross-links were mapped to a high-resolution structure of BSA (PDB: 4F5S). (B) Distance distribution plot of TSTO cross-links, 90% of which were satisfied with Cα-Cα distances below the threshold of 35 Å.

FIG. 6A-F presents representative MSⁿ analyses of the three types of TSTO cross-links identified from 26S proteasomes. (A) MS² fragmentation of a tripeptide tri-link [α, β, γ] (m/z 998.9354⁵⁺) yielded a series of dominant ion doublets corresponding to α_{AR}/α_{AR*}, β_{AR}/β_{AR*}, and γ_{AR}/γ_{AR*} peptides. (B) MS³ analyses of the cross-link fragments α_{AR*} (m/z 652.3762²⁺), β_{AR*} (m/z 895.4729²⁺), and γ_{AR*} (m/z 922.9833²⁺) identified them as ¹¹⁰YIINVK_{AR*}QFAK¹²⁰ [SEQ ID NO:8], ¹⁹⁸VVSSSIVDK_{AR*}YIGESAR²¹³ [SEQ ID NO:9], and ²³⁰VVGSEFVQK_{AR*}YLGEGPR²⁴⁵ [SEQ ID NO:10], signifying a tripeptide tri-link among Rpt1:K116, Rpt4:K206, and Rpt3:K238. (C) MS² fragmentation of a dipeptide tri-link [α2-β₂] (m/z 1209.0961⁴⁺) yielded two dominant sets of ions—a doublet representing α_{AR*}/α_{AR} and a triplet representing β_{2AR}/β_{AR_AR*}/β_{2AR*} peptides. (D) MS³ analyses of the cross-link fragments α_{AR*} (m/z 761.4022²⁺) and β_{2AR*} (m/z 1086.8612²⁺) identified their sequences as ²⁷⁸AYEK_{AR*}ILFTEATR²⁸⁹ [SEQ ID NO:11] and ⁴⁴⁴DGVIEASINHEK_{AR*}GYVQ SK_{AR*}EMIDIYSTR⁴⁷⁰ [SEQ ID NO:12] respectively, signifying a dipeptide tri-link among Rpn12:K281, Rpn3:K455, and Rpn3:K461. (E) MS² fragmentation of a dipeptide bi-link [α-β] (m/z 773.4180⁴⁺) resulted in two dominant ion doublets α_{AR}/α_{AR*} and β_{AR}/β_{AR*}. (F) MS³ analyses of the cross-link fragments α_{AR*} (m/z 591.8356²⁺) and β_{AR*} (m/z 878.9664²⁺) identified their sequences as ⁶⁹FIVK_{AR*}ATNGPR⁷⁸ [SEQ ID NO:13] and ²¹⁴VSGSELVQK_{AR*}FIGEGAR²²⁹ [SEQ ID NO:14], signifying a pair-wise cross-link between Rpt4:K72 and Rpt6:K222. Note: AR: aldehyde remnant moiety; AR*: aldehyde remnant moiety after water loss (i.e., AR-H₂O).

FIG. 7A-E demonstrates TSTO cross-linking of human 26S proteasome. (A) XL-PPI map of 26S cross-links. In total, 32 subunits were identified participating in inter-subunit interactions-19 from the 19S subcomplexes and 13 from the 20S subcomplex. 19S lid subunits shown in blue, 19S base subunits in dark grey, 20S α ring subunits in yellow, and 20S β ring subunits shown in light grey. Medium grey lines represent interactions that have been captured as tri-links; grey lines represent other cross-links. Mapping of trimeric interactions to a high-resolution 26S proteasome structure: (B) lining the inner pore between Rpt subunits of the AAA-ATPase ring (19S base subcomplex), (C, D) Interactions describing connectivity of 19S base subcomplex subunits to the 20S, and (E) Dss1 with 19S lid subunits Rpn3 and Rpn7.

FIG. 8A-C demonstrates Dss1 interactions captured by TSTO cross-linking. (A) 2-D XL-map of Dss1 cross-links to 19S lid subunit Rpn3, Rpn6, and Rpn7. Trimeric interactions are highlighted in red. Dss1-Rpn3, Dss1-Rpn6, and Dss1-Rpn7 cross-links were mapped to the high-resolution 26S proteasome structures: (B) PDB:7QY7 and (C) PDB:6MSB.

FIG. 9 presents distance distribution plots of unique residue pairs derived from TSTO cross-linking of 26S proteasomes. Unique residue pairs from TSTO bi-links are shown in gray, while those from TSTO tri-links are shown in light blue. Cross-linked residues were mapped onto a high-resolution structure of the 26S proteasome (PDB: 7QY7).

FIG. 10A-D presents integrative structural modeling of the base subcomplex of the human 26S proteasome using synthetic and experimental XL data. (A) Model accuracy and (B) cluster precision was assessed as a function of the number of cross-links, using six replicates per condition with synthetic bifunctional and trifunctional cross-linker data. Black lines indicate mean values for each subset. (C) Model accuracy and (D) cluster precision were compared for models generated using TSTO (18 trivalent+65 bivalent) and DSSO (83 bivalent) cross-linking data. For experimental TSTO data in (B) and (D), five datasets were generated, each combining all 18 trivalent with 65 randomly selected (from 143 available) bivalent cross-links.

FIG. 11A-B provides evaluation and optimization of TSTO in vivo cross-linking. TSTO cross-linking was tested at various concentrations (0.5-3 mM) using HEK 293^{HBTH-CSN2} cells. The cross-linked products were separated by SDS-PAGE, transferred onto a PVDF membrane, and evaluated by (A) amido black staining and (B) western blot analysis using StrepHRP to probe HBTH-tagged CSN2. The bands corresponding to the oligomers and monomer of HBTH-tagged CSN2 were indicated.

FIG. 12 presents an in vivo TSTO cross-linking workflow.

FIG. 13A-C provides 26S TSTO cross-linking analysis. (A) Venn diagrams depicting the overlap of inter- and intra-protein PPIs described by TSTO bi- and tri-links captured from in vivo cross-linking. (B) Histogram of mapped Ca—Ca distances for 1790 URPs across 539 CORUM complexes; 95% were found to be ≤35 Å. (C) In vivo XL-PPI network of HEK 293 cells derived from TSTO cross-links comprising 1512 nodes connected by 1242 edges.

FIG. 14A-B demonstrates evaluation of the TSTO XL-Proteome. (A) Gene Ontology (GO) analysis showing cell compartment distribution of TSTO XL-proteome compared to GO proteome and DSBSO XL-proteome. In both cross-linking datasets, cytosolic proteins are enriched, and plasma membrane proteins have decreased representation compared to the Gene Ontology proteome. (B) Distribution of STRING scores for XL-PPIs captured by in vivo TSTO cross-linking compared to human PPIs curated within the STRING database.

FIG. 15 provides a comparison of protein abundances across the TSTO XL-proteome, MS-proteome and several published XL-proteomes. Protein abundance distribution of the human cell MS proteome determined by shotgun proteomics (dark-medium grey) compared to selected XL-proteomes (alkyne-A-DSBSO in vivo dataset (medium-light grey), tBu-PhoX in vivo dataset (dark grey), DSSO in vitro dataset (light grey), and TSTO in vivo dataset from this work (grey)).

FIG. 16 demonstrates mapping of TSTO cross-links to a high-resolution structure of the 80S ribosomal complex (PDB: 6Z6M). Mapped distances between residues identified in trimeric cross-links are shown in dark grey, while other cross-links are shown in medium grey. The overall satisfaction rate of mapped cross-links ≤35 Å was 96%.

FIG. 17A-C demonstrates mapping of homotrimeric TSTO cross-links from in vivo XL-MS. (A) Tri-link through K100 residues of three NME2 proteins, mapped to a high-resolution structure (PDB: 8PYW). (B) Trimeric cross-links involving residues K54 and K56 of three HSPE1 proteins mapped to a high-resolution structure (PDB:4PJ1). (C) Tri-link through K262 of three ATAD3a proteins, mapped to an AlphaFold3-generated model for an ATAD3a trimer.

FIG. 18 provides TSTO XL-MS data of mouse heart tissue. Interaction network identified by TSTO cross-linking.

FIG. 19A-D presents an analysis of mouse heart tissue cross-linking. (A) XL-PPI network describing interactions among proteins associated with heart muscle function. (B-D) GO enrichment analysis of TSTO XL-proteome describing (B) molecular function, (C) cellular components, and (D) biological processes associated with heart-specific and enriched proteins.

### DETAILED DESCRIPTION

As used herein and in the appended claims, the singular forms “a,” “an,” and “the” include plural referents unless the context clearly dictates otherwise. Thus, for example, reference to “a crosslinker” includes a plurality of such crosslinkers and reference to “the sulfoxide group” includes reference to one or more sulfoxide groups and equivalents thereof known to those skilled in the art, and so forth.

It is to be further understood that where descriptions of various embodiments use the term “comprising,” those skilled in the art would understand that in some specific instances, an embodiment can be alternatively described using language “consisting essentially of” or “consisting of.”

As used herein, “about” means a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “alkenyl”, as used in this disclosure, refers to an organic group that is comprised of carbon and hydrogen atoms that contains at least one double covalent bond between two carbons. Typically, an “alkenyl” as used in this disclosure, refers to organic group that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkenyl can form a double bond to a carbon of a parent chain, an alkenyl group of three or more carbons can contain more than one double bond. It certain instances the alkenyl group will be conjugated, in other cases an alkenyl group will not be conjugated, and yet other cases the alkenyl group may have stretches of conjugation and stretches of nonconjugation. Additionally, if there is more than 2 carbon, the carbons may be connected in a linear manner, or alternatively if there are more than 3 carbons then the carbons may also be linked in a branched fashion so that the parent chain contains one or more secondary, tertiary, or quaternary carbons. An alkenyl may be substituted or unsubstituted, unless stated otherwise.

The term “alkyl”, as used in this disclosure, refers to an organic group that is comprised of carbon and hydrogen atoms that contains single covalent bonds between carbons. Typically, an “alkyl” as used in this disclosure, refers to an organic group that contains 1, 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. Where if there is more than 1 carbon, the carbons may be connected in a linear manner, or alternatively if there are more than 2 carbons then the carbons may also be linked in a branched fashion so that the parent chain contains one or more secondary, tertiary, or quaternary carbons. An alkyl may be substituted or unsubstituted, unless stated otherwise.

The term “alkynyl”, as used in this disclosure, refers to an organic group that is comprised of carbon and hydrogen atoms that contains a triple covalent bond between two carbons. Typically, an “alkynyl” as used in this disclosure, refers to organic group that contains that contains 2, 3, 4, 5, 6, 7, 8, 9, 10, 11, 12, 13, 14, 15, 16, 17, 18, 19, 20, or 30 carbon atoms, or any range of carbon atoms between or including any two of the foregoing values. While a C₂-alkynyl can form a triple bond to a carbon of a parent chain, an alkynyl group of three or more carbons can contain more than one triple bond. Where if there is more than 3 carbon, the carbons may be connected in a linear manner, or alternatively if there are more than 4 carbons then the carbons may also be linked in a branched fashion so that the parent chain contains one or more secondary, tertiary, or quaternary carbons. An alkynyl may be substituted or unsubstituted, unless stated otherwise.

The term “aryl”, as used in this disclosure, refers to a conjugated planar ring system with delocalized pi electron clouds that contain only carbon as ring atoms. An “aryl” for the purposes of this disclosure encompasses from 1 to 4 aryl rings wherein when the aryl is greater than 1 ring the aryl rings are joined so that they are linked, fused, or a combination thereof. An aryl may be substituted or unsubstituted, or in the case of more than one aryl ring, one or more rings may be unsubstituted, one or more rings may be substituted, or a combination thereof.

The term generally represented by the notation “Cₓ-C_{y}” (where x and y are whole integers and y>x) prior to a functional group, e.g., “C₁-C₁₂ alkyl” refers to a number range of carbon atoms. For the purposes of this disclosure any range specified by “Cₓ-C_{y}” (where x and y are whole integers and y>x) is not exclusive to the expressed range but is inclusive of all possible ranges that include and fall within the range specified by “Cₓ-C_{y}” (where x and y are whole integers and y>x). For example, the term “C₁-C₄” provides express support for a range of 1 to 4 carbon atoms, but further provides implicit support for ranges encompassed by 1 to 4 carbon atoms, such as 1 to 2 carbon atoms, 1 to 3 carbon atoms, 2 to 3 carbon atoms, 2 to 4 carbon atoms, and 3 to 4 carbon atoms.

The term “functional group” or “FG” refers to specific groups of atoms within molecules that are responsible for the characteristic chemical reactions of those molecules. While the same functional group will undergo the same or similar chemical reaction(s) regardless of the size of the molecule it is a part of, its relative reactivity can be modified by nearby functional groups. The atoms of functional groups are linked to each other and to the rest of the molecule by covalent bonds. Examples of FGs that can be used in this disclosure, include, but are not limited to, halogens, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, sulfhydryls, sulfides, disulfides, sulfinyls, sulfos, thiocyanates, isothiocyanates, carbonothioyls, phosphinos, phosphonos, and phosphates.

The term “optionally substituted” refers to a functional group, typically a hydrocarbon or heterocycle, where one or more hydrogen atoms may be replaced with a substituent. Accordingly, “optionally substituted” refers to a functional group that is substituted, in that one or more hydrogen atoms are replaced with a substituent, such as a FG, or unsubstituted, in that the hydrogen atoms are not replaced with a substituent. For example, an optionally substituted hydrocarbon group refers to an unsubstituted hydrocarbon group or a substituted hydrocarbon group.

The term “substituent” refers to an atom or group of atoms substituted in place of a hydrogen atom. For purposes of this invention, a substituent would include deuterium atoms. Examples of substituents that can replace a hydrogen group in the structure of a crosslinker disclosed herein include, but are not limited to, halogen, hydroxyl, carboxyl, aldehyde, nitrile, isonitrile, nitro, amino, sulfide, alkyl (e.g., (C₁-C₆)alkyl), alkenyl (e.g., (C₁-C₆)alkenyls), alkynyl (e.g., (C₁-C₆)alkynyl), alkoxy (e.g., (C₁-C₆) alkoxy, ester (e.g., (C₁-C₆) ester), aryl, cycloalkyl, and heterocycle.

The term “substituted” with respect to hydrocarbons, heterocycles, and the like, refers to structures wherein the parent chain contains one or more substituents.

The term “unsubstituted” with respect to hydrocarbons, heterocycles, and the like, refers to structures wherein the parent chain contains no substituents.

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated by reference in full for the purpose of describing and disclosing methodologies that might be used in connection with the description herein. The publications are provided solely for their disclosure prior to the filing date of the present application. Nothing herein is to be construed as an admission that the inventors are not entitled to antedate such disclosure by virtue of prior disclosure. Moreover, with respect to any term that is presented in one or more publications that is similar to, or identical with, a term that has been expressly defined in this disclosure, the definition of the term as expressly provided in this disclosure will control in all respects.

Cross-linking mass spectrometry (XL-MS) has emerged as a transformative technology for elucidating the structure, interactions, and dynamics of proteins. By covalently linking proximal amino acid residues within or between proteins, XL-MS provides unique insights into the architecture of protein complexes and the spatial arrangement of individual protein domains. Due to various innovations, this methodology has revolutionized structural biology by complementing traditional structural techniques and offering a more holistic view of protein assemblies in their native environments. In particular, diverse MS-cleavable cross-linking reagents have been developed to facilitate the detection and identification of cross-linked peptides, enabling effective proteome-wide analyses of cellular networks to elucidate their structural organization from intact cells, subcellular organelles, tissues and clinical samples.

As the most commonly used cross-linking reagents are homo- and/or heterobifunctional cross-linkers, current XL-MS analyses have prioritized the identification of peptides cross-linked between a pair of residues to infer pair-wise interactions and delineate PPI physical contacts. While multimeric cross-links can be formed using bifunctional cross-linkers, the difficulty of their identification has rendered them largely invisible. Due to the additional expansion of search space associated with each successive peptide, traditional non-cleavable cross-linking reagents are incapable of this task. As a result, MS-cleavable cross-linking reagents are critical for alleviating this issue by facilitating the physical separation of cross-linked constituents for individual identification. Even so, the additional bonds required for cross-linking each successive peptide complicate cross-link fragmentation by yielding excessive cleavage products and introducing ambiguity in designating cross-linked sites. During the course of the study, a homotetrafunctional MS-cleavable cross-linking reagent utilizing four NHS ester-targeting groups was very recently reported. The four MS-cleavable DP bonds—one for each NHS ester-containing arm—can fragment to separate all cross-linked peptides in MS², allowing individual peptides to be sequenced in subsequent MS³. However, the fragmentation appears to be complex due to incomplete cleavage of all DP bonds—as well as simultaneous peptide backbone fragmentation. While ion fragment selection for MS³ from the resulting convoluted MS² spectra has been addressed using real-time MS acquisition methods, the heterogeneity and low abundance of cross-linked fragments remains a hindrance for analysis of higher-order cross-links. Therefore, it would be advantageous to implement an MS-cleavable bond that is weaker than peptide bonds in the design of multifunctional cross-linkers to minimize the number of fragment ions for subsequent MS³ analysis.

To this end, the disclosure provides a homo-multifunctional trioxane based MS-cleavable cross-linker that simultaneously targets multiple residues, but more importantly streamlines the peptide identification process by utilizing a core structure that elegantly and fully releases all cross-linked peptides in a single step. The trioxane based MS-cleavable cross-linker of the disclosure expands the detection of protein connectivity and provides additional restraint information for improved structural elucidation.

Provided herein is the development of novel trioxane-based MS cleavable cross-linkers that enable simultaneous capture of multimeric PPIs. In particular, provided herein is the design, synthesis and characterization of a novel membrane-permeable, MS-cleavable homotrifunctional cross-linker, TSTO (tris-succinimidyl trioxane) to enable simultaneous capture and identification of trimeric PPIs. XL-MS analysis of human 26S proteasomes has demonstrated that the novel trioxane-based MS cleavable cross-linkers of the disclosure (e.g., TSTO) are effective in cross-linking protein complexes and that the resulting cross-linked peptides display unique and predicable fragmentation during collision-induced dissociation (CID), enabling their simplified and accurate identification using multistage mass spectrometry (MSⁿ). Importantly, the trioxane-based MS cleavable cross-linkers disclosed herein captured trimeric interactions to better define interfaces between proteasome subcomplexes. Additionally, the trioxane-based MS cleavable cross-linkers disclosed herein (e.g., TSTO) have been successfully applied for in vivo cross-linking of HEK293 cells and mouse heart tissues, demonstrating its applicability in elucidating cellular networks. Apart from binary interactions, trimeric interactions captured by the trioxane-based MS cleavable cross-linker of the disclosure facilitated the characterization of protein oligomers and enhanced structural modeling with improved accuracy and precision. In summary, it was shown in the studies presented herein, that the trioxane-based MS cleavable cross-linkers of the disclosure can uncover multimeric interactions to yield more detailed PPI networks, advancing our understanding of cellular processes and biological function.

In a particular embodiment, a trioxane-based MS cleavable cross-linker disclosed herein includes three cross-linking arms. By having three cross-linking arms the trioxane-based MS cleavable cross-linker can simultaneously capture and identify trimeric PPIs. In yet a further embodiment, the trioxane-based MS cleavable cross-linker has a unique symmetrical structure comprising three NHS esters connected via a central trioxane group, permitting concurrent cross-linking between three lysine residues to form a trimeric cross-link among three individual peptides (aka tripeptide tri-link). In comparison to a traditional cross-link between two individual peptides, accurate identification of a tri-link would be much more challenging due to further expansion of database search space (n³). Therefore, the design of the central trioxane is important as can have three equal MS-cleavable bonds that are weaker than peptide bonds and can be cleaved using collision-induced dissociation (CID) to simultaneously release all three cross-linking arms.

While MS²-based data acquisitions have become popular for analyzing MS-cleavable dipeptide cross-links, it is envisaged that MS³-based approaches would be preferred for the identification of tripeptide cross-links. The co-fragmentation of three peptides within a single spectrum heavily convolutes database searching, impeding identification of higher-order cross-linked species by MS²-based approaches. Moreover, the central trioxane presents an innovative core structure for developing multifunctional MS-cleavable cross-linkers as one arm can be replaced with other functional groups-such as enrichment, isobaric, or reporter tags—to enable cross-link purification, quantitation, or further improve cross-link detection and identification. While, the exemplary trioxane-based MS cleavable cross-linker disclosed herein, TSTO, comprises NHS ester groups, it should be understood that the NHS ester groups can be replaced by other reactive chemistries to target specific or non-specific amino acids, expanding the coverage of XL proteomes. As with TSTO, the cleavage of the trioxane within the mass spectrometer would release any additional functional groups, preventing their impact on cross-linked peptide identification. Thus, the development of TSTO presented here opens a new direction for designing diverse cross-linkers to further advance XL-MS technologies.

In a particular embodiment, the disclosure provides a trioxane-based mass spectrometry (MS)-cleavable cross-linker comprising: a central trioxane group, which may be isotopically enriched with heavier isotopes selected from C¹³, and O¹⁸; two or more MS-cleavable bonds; two or more reactive cross-linking groups that can react with amino acids of peptides and/or proteins; optionally, linker arms that connect the reactive cross-linking groups with the central trioxane group, wherein the linker arms may be isotopically enriched with heavier isotopes selected from H², C¹³, O¹⁸ and N¹⁵. In a further embodiment, the trioxane-based mass spectrometry MS-cleavable cross-linker is configured to form a dimeric or trimeric cross-links with amino acids of peptides and/or proteins. In another embodiment, the two or more MS-cleavable bonds can be cleaved using collision-induced dissociation. In yet another embodiment, the two or more reactive cross-linking groups are located equal distant to the central trioxane group. In a further embodiment, the two or more reactive cross-linking groups are selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative.

In a certain embodiment, a trioxane-based MS cleavable cross-linker disclosed herein can be coupled with isotope-based quantitative strategies to enable the interrogation of how multimeric protein interactions fluctuate in response to biological stimuli, disease states, or drug treatments, providing a deeper functional understanding of cellular organization. The isotopic labels can be introduced into trioxane-based cross-links by SILAC labeling of lysines, chemical labeling of cross-linked peptides with isobaric reagents (e.g., TMT), or coding isotopic labels in the linker design. Isotope coding in crosslinking uses different isotopic forms (e.g., heavy and light isotopes) of a crosslinker to distinguish crosslinked peptides in mass spectrometry. This allows for easier identification of interacting peptides and precise localization of the crosslink site Accordingly, for various structures making up a trioxane-based mass MS-cleavable cross-linker disclosed herein, light isotopes can be replaced by heavier isotopes (such as H¹->H², O¹⁶->O¹⁸, N¹⁴->N¹⁵, and/or C¹²->C¹³) to generate isotope coded cross-links. Different combinations of isotope incorporation allow for generation of structures that have same composition of elements but have different masses, which can then be used for quantitation at MS¹ level. Alternatively, a trioxane-based mass spectrometry (MS)-cleavable cross-linker that comprises identical structures and masses. The resulting linkers will be isobaric cross-linkers which can be used for quantitation at MS² level. With these linkers, the cross-linked peptides have the same mass in MS¹, which will yield fragment ions in MS² with different masses for quantitation. In yet a further embodiment, the central trioxane group comprises one or more of C¹³ and/or O¹⁸ atoms. In a certain embodiment, the trioxane-based mass spectrometry (MS)-cleavable cross-linker comprise 2 or more linker arms that connect the reactive cross-linking groups with the central trioxane group, wherein the linker arms may be isotopically enriched with heavier isotopes selected from H², C¹³, O¹⁸ and N¹⁵. In another embodiment, one or more of the linker arms comprise one or more of H², C¹³, O¹⁸ and/or N¹⁵ atoms. In another embodiment, one of the reactive cross-linking groups comprises one or more of H², C¹³, O¹⁸ and/or N¹⁵ atoms. In another embodiment, two of the reactive cross-linking groups comprises one or more of H², C¹³, O¹⁸ and/or N¹⁵ atoms.

Additionally, the disclosure also provides for a trioxane-based mass MS-cleavable cross-linker disclosed herein that comprises an enrichable handle in the place of one of the reactive cross-linking groups. In such a situation, the trioxane-based mass MS-cleavable cross-linker would comprise two reactive cross-linking groups and 1 enrichable handle group. “Enrichable handles” as used herein, refer to chemical groups or tags attached to the central trioxane groups, either directly or via a linker arm, to facilitate purification, detection or analysis of cross-linked products. This strategy allows for the selective isolation and enrichment of cross-linked products from complex biological samples. Enrichable handles typically utilize a specific binding interaction or chemical reaction to isolate the target molecules. For instance, a handle could be an affinity tag that binds to a specific resin or a chemical group that allows for a ‘click chemistry’ reaction. Examples of enrichable handles used in purification: Phosphonate-handles (PhosID): These handles, containing a stable P—C bond, can be used for enrichment of cross-linked peptides through immobilized metal affinity chromatography (IMAC). Biotin: Biotin is a commonly used affinity tag that interacts strongly with avidin or streptavidin, enabling efficient enrichment of cross-linked products tagged with biotin. Phosphonic acid handle (in PhoX crosslinker): The PhoX crosslinker incorporates a phosphonic acid handle, which allows for the enrichment of cross-linked peptides via IMAC. This enhances the detection and identification of cross-linked peptides in mass spectrometry studies. Azide or Alkyne groups (in crosslinkers): These groups allow for “click chemistry” based enrichment strategies. This involves using these groups to attach an affinity tag (like biotin) after the crosslinking reaction, followed by affinity enrichment. An antibody that binds to the enrichable handle is also contemplated herein. The benefits of a trioxane-based mass MS-cleavable cross-linker disclosed herein comprising an enrichable handle are many, including but not limited to, enrichment of cross-linked products, increased sensitivity, reduced background noise, simplified workflows, and enhanced analytical depth. In a particular embodiment, a trioxane-based MS cleavable cross-linker disclosed herein comprises two cross-linking arms and one arm that does not form cross-links. This non-cross-linking arm could have a low molecular weight and thus not contribute to complexity in the MS/MS data. Alternatively, the non-cross-linking arm can contain a functional group such as biotin for affinity purification. Another benefit of the trioxane-based MS cleavable cross-linker disclosed herein is that the two cross-linking arms could be designed to be of equal mass, so it would not matter which orientation the cross-linker reacted with the protein as the two expected fragments of the cross-linking arms would have the exact mass. In a particular embodiment, the trioxane-based mass spectrometry (MS)-cleavable cross-linker disclosed herein comprises 2 linker arms that connect the reactive cross-linking groups with the central trioxane group, and comprise a linker arm that connects an enrichable handle or a fluorophore to the central trioxane group. In a further embodiment, the enrichable handle is selected from a click chemistry linker, a phosphate, a phosphonate, a fluorophore, biotin, an azide, an alkyne, and a phosphonic acid. In yet a further embodiment, the trioxane-based mass spectrometry (MS)-cleavable cross-linker comprise 2 linker arms that connect the reactive cross-linking groups with the central trioxane group, and comprise a linker arm that connects an enrichable handle or a fluorophore to the central trioxane group.

The disclosure also provides that the trioxane-based mass spectrometry MS-cleavable cross-linker disclosed herein, based upon the choice of the reactive cross-linking groups may be homotrifunctional, homobifunctional, or heterotrifunctional, or hereterobifunctional. Additionally, as noted above, the reactive cross-linking groups can be isotope-coded as noted above. In a particular embodiment, the trioxane-based mass spectrometry (MS)-cleavable cross-linker comprises 3 linker arms that connect three reactive cross-linking groups with the central trioxane group. In yet another embodiment, the three reactive cross-linking groups have different structures, or have different atomic masses. In a further embodiment, two of the reactive cross-linking groups have the same structure, and one of the reactive cross-linking groups has a different structure or has a different atomic mass. In yet a further embodiment, all three of the reactive cross-linking groups have the same structure. For each of the foregoing embodiments, the various groups may be isotope-coded as noted above.

In a particular embodiment, the disclosure provides for a trioxane-based mass spectrometry (MS)-cleavable cross-linker that has the structure of:

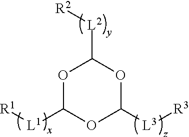

wherein, L¹, L², and L³ are linker arms each individually selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide,

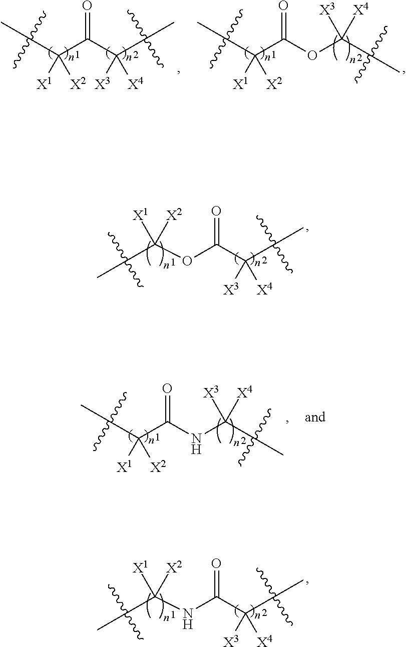

wherein L¹, L², and L³ may be isotopically enriched with heavier isotopes selected from H², C¹³, O¹⁸ and N¹⁵; R¹-R³ are individually selected from a reactive cross-linking group that can react with amino acids of peptides and/or proteins, an enrichable handle, and a fluorophore, wherein at least two of R¹ to R³ are reactive cross-linking groups; X¹-X⁴ are each individually selected from FG, H, (C₁-C₆)alkyl, (C₁-C₆)alkenyl, (C₁-C₆)alkynyl, cyano, azide, hydroxyl, aldehyde, carboxyl, halo, amide, and amine, wherein each of the foregoing groups may be isotopically enriched with heavier isotopes selected from H², C¹³, O¹⁸ and/or N¹⁵; x, y, and z are integers selected from 0 and 1; and n¹ and n² are integers selected from 0, 1, 2, 3, 4, 5 and 6. In a further embodiment, the trioxane-based MS-cleavable cross-linker is configured to form dimeric or trimeric cross-links with amino acids of peptides and/or proteins. In yet a further embodiment, L¹ to L³ are individually selected from optionally substituted (C₁-C₆)alkyl,

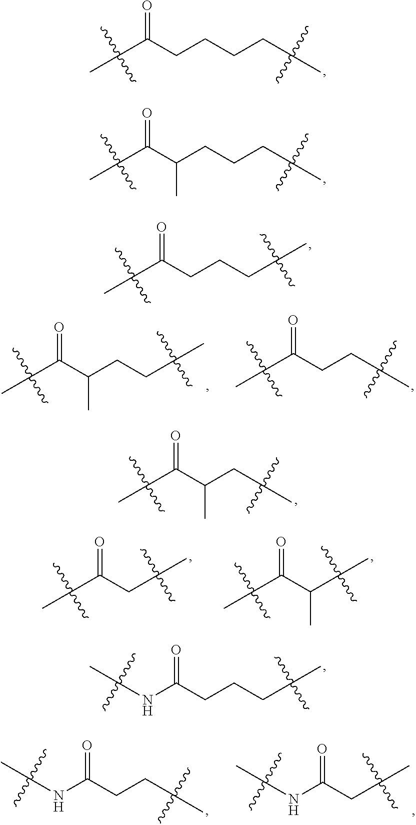

wherein each of the foregoing groups may be isotopically enriched with heavier isotopes selected from H², C¹³, O¹⁸ and N¹⁵. In another embodiment, L¹ to L³ are

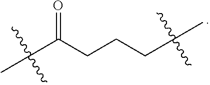 [SMILES: *CCCC(*)=O]

In yet another embodiment, the reactive cross-linking group is selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative. In a further embodiment, for R¹, R², and R³ the reactive cross-linking groups that can react with amino acids of peptides and/or proteins are selected from

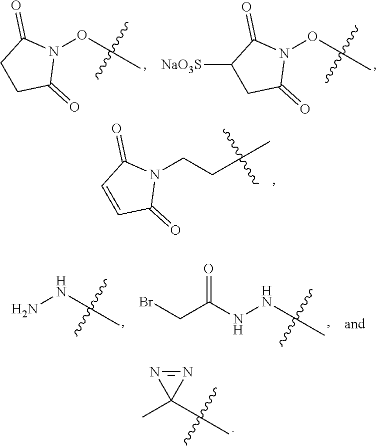

In a further embodiment, the enrichable handle for R¹, R², and R³ is selected from a click chemistry linker, a phosphate, a fluorophore, biotin, an azide, an alkyne, and a phosphonic acid. In yet a further embodiment, R¹, R², and R³ are reactive cross-linking groups, and wherein: (i) at least two of R¹, R², and R³ have same structure, or (ii) R¹, R², and R³ have same structure, or (iii) R¹, R², and R³ have different structures.

In a certain embodiment, the disclosure provides for a trioxane-based mass spectrometry (MS)-cleavable cross-linker that has the structure of:

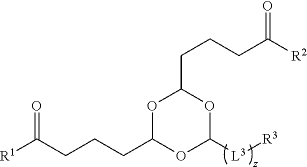

wherein, L³ is a linker arm selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide,

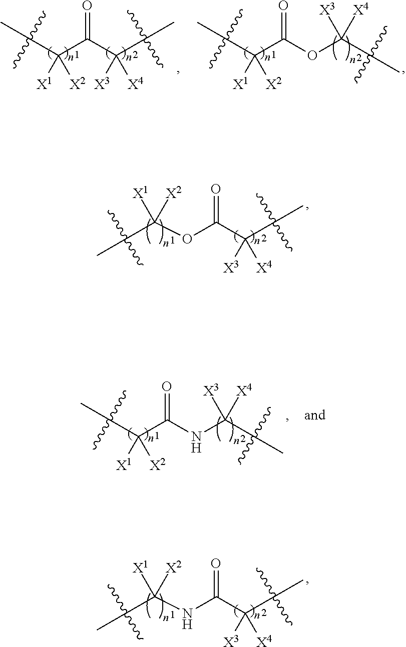

wherein L³ may be isotopically enriched with heavier isotopes selected from H², C¹³, O¹⁸ and N¹⁵; R¹ and R² are

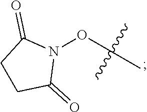 [SMILES: *ON1C(=O)CCC1=O]

R³ is selected from an enrichable handle, a fluorophore,

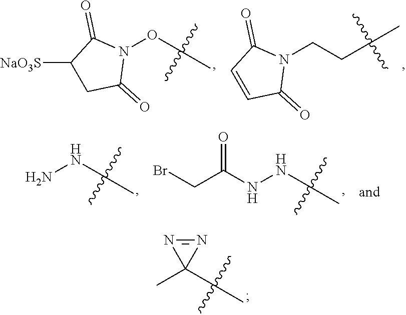

z is an integer selected from 0 and 1; and n¹ and n² are integers selected from 0, 1, 2, 3, 4, 5 and 6. In a certain embodiment, the MS-cleavable trioxane-based cross-linker has the structure of:

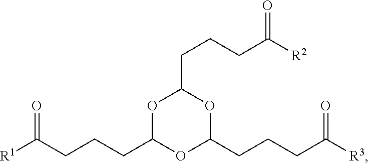

wherein, R¹-R³ are each individually selected from

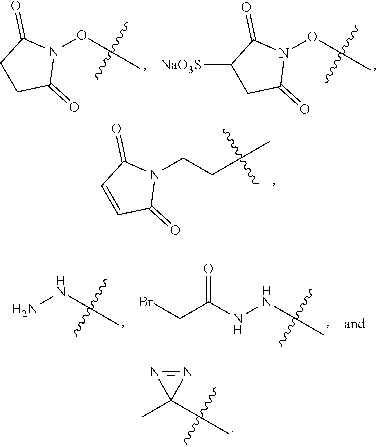

In a particular embodiment, a trioxane-based mass spectrometry (MS)-cleavable cross-linker disclosed herein is tris-succinimidyl trioxane (TSTO) having the structure of:

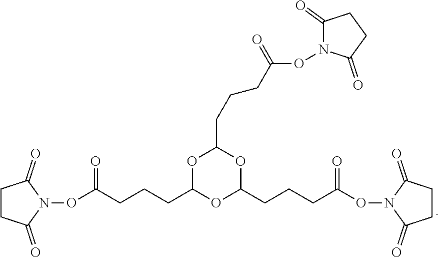 [SMILES: O=C(CCCC1OC(CCCC(=O)ON2C(=O)CCC2=O)OC(CCCC(=O)ON2C(=O)CCC2=O)O1)ON1C(=O)CCC1=O]

The disclosure provides for the successful development and employment of the trioxane-based XL-MS platform to capture trimeric interactions of protein complexes and cellular networks from intact cells and tissues, yielding structural details that cannot be easily obtained by existing reagents. The information obtained can be combined with AlphaFold prediction and/or integrative structural modeling to enhance the characterization of cellular networks in future studies. Therefore, the trioxane-based XL-MS platform of the disclosure represents a highly promising approach for advancing XL-MS technology towards systems structural biology in vivo.

Compared to cross-linking reagents that target two residues, the trioxane-based MS cleavable cross-linkers of the disclosure enhances the localization of interacting proteins by simultaneously targeting three residues, triangulating a multi-point attachment that provides greater spatial resolution and allows more precise mapping of protein interfaces and interaction sites. This is particularly useful for modeling multimeric interactions, especially for protein complexes that can exist in different compositional states. As bifunctional cross-linkers can only provide spatial restraints between two residues, it can be difficult to correctly assign them to individual compositional forms without reiterative modeling. Therefore, trivalent cross-links offer an additional restraint to help facilitate characterization of multi-protein interactions. As shown with cross-linking of the 26S proteasome, TSTO has identified trimeric interactions bridging subunits of different proteasome subcomplexes that accurately describe their positioning within the 26S holocomplex—such as those between the 19S lid and base, and the 19S base and 20S subcomplexes. In addition, the tri-links have accurately described the orientation of multi-subunit interactions within their respective subcomplexes, such as the localization of Dss1 to the outer 19S lid and the close proximities of solvent-accessible residues of the ATPase ring subunits lining the central pore of the 19S base. Moreover, integrative structural modeling of a proteasome subcomplex has demonstrated that trivalent cross-links are advantageous in providing additional spatial information to facilitate structure modeling with increased precision and accuracy. This analysis has established a solid foundation for us to better characterize structural organization of cellular networks in the future.

Similarly, the trioxane-based MS cleavable cross-linkers disclosed herein, like TSTO, have identified trimeric interactions from in vivo XL-MS analyses, for instance triangulating the positions of ribosome-interacting proteins relative to the larger 80S ribosomal machinery, as well as various subunit interfaces within and between its 40S and 60S subcomplexes. In addition to revealing the identities of heteromeric interactions, trioxane-based MS cleavable cross-linkers of the disclosure expand the capability of XL-MS to identify homomeric ones. While bifunctional cross-linkers can reveal homodimer formation by identifying cross-linked peptides with overlapping sequences, trioxane-based MS cleavable cross-linkers disclosed herein are capable of placing homodimeric interactions in the three-dimensional context of multiprotein assemblies by capturing a third residue, providing a clearer picture of how individual dimers are oriented as part of a larger protein complex. Furthermore, the trioxane-based MS cleavable cross-linkers of the disclosure can unravel homotrimeric interactions—as confirmed for NME2 and HSPE1—which can be used in conjunction with structure prediction such as AlphaFold to model unknown structures.

Accordingly, the disclosure also provides methods or processes for mapping intra-protein interactions in a protein, inter-protein interactions in a protein complex, or any combination thereof. In a particular embodiment, the method comprises: contacting the protein and/or the protein complex with a trioxane-based MS cleavable cross-linker disclosed herein to form a cross-linked product; digesting the cross-linked product to form a plurality of fragments, wherein a portion of the plurality of fragments comprises cross-linked peptide fragments; and identifying and analyzing cross-linked peptide fragments using tandem mass spectrometry (MSⁿ) to map intra-protein interactions in the protein and/or inter-protein interactions in the protein complex. In a further embodiment, the MS-cleavable trioxane-based cross-linker is used at a molar ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1, or a range that includes or is between any two of the foregoing ratios (e.g., 1:5 to 5:1), to the protein and/or the protein complex. In a further embodiment, the MS-cleavable trioxane-based cross-linker is used at a molar ratio of 1:5 to 5:1 to the protein and/or the protein complex. In yet a further embodiment, the cross-linked product was digested with one or more proteases. Examples of proteases include, but are not limited to, serine proteases, threonine proteases, aspartic proteases, glutamic proteases, metalloproteases, and asparagine peptide lyases. In a particular embodiment, the one or more proteases are serine proteases. In another embodiment, a data-dependent MS³ acquisition method is used for identifying and analyzing the cross-linked peptide fragments. In yet another embodiment, the results of the data-dependent MS³ acquisition method is used with a computer program that preforms predictions of protein structure. In a further embodiment, the computer program is an artificial intelligence-based program. In yet a further embodiment, the computer program is a AlphaFold based program.

The disclosure also provides methods for mapping global protein-protein interactions (PPIs) from a sample comprising a plurality of proteins using a trioxane-based MS cleavable cross-linker disclosed herein. In a particular embodiment, a method for mapping global protein-protein interactions (PPIs) from a sample comprising a plurality of proteins comprises: contacting the sample comprising a plurality of proteins with the trioxane-based MS cleavable cross-linker disclosed herein to form crosslinked proteins; digesting the crosslinked proteins to form crosslinked protein fragments or peptides; isolating fractions that are enriched with cross-linked protein fragments or peptides in the sample; analyzing the fractions using tandem mass spectrometry (MSn) and protein database searching to identify cross-linked protein fragments or peptides; and mapping the identified cross-linked protein fragments or peptides to generate a global structural map of PPIs. In a further embodiment, the sample is a cellular sample or a tissue sample. In yet a further embodiment, the cross-linked product is digested with one or more proteases. Examples of proteases include, but are not limited to, serine proteases, threonine proteases, aspartic proteases, glutamic proteases, metalloproteases, and asparagine peptide lyases. In another embodiment, the one or more proteases are serine proteases. In a further embodiment, a data-dependent MS³ acquisition method is used for identifying the cross-linked protein fragments or peptides. In yet a further embodiment, the identified cross-linked protein fragments or peptide are mapped using various databases or programs that profile protein to protein interactions. Examples of databases or programs that profile protein to protein interactions, include, but are not limited to, AlphaFold, APID, MiMI, iRefindex, String, BioGrid, HPIDB, MINT, DIP, IntAct, HPRD, CORUM, and BioPlex. In yet another embodiment, the methods for mapping global protein-protein interactions (PPIs) further comprises the use of one or more additional MS-cleavable cross-linkers (e.g., BMSO, DBrASO, DSSO, DHSO, SDASO-S, Azide-A-DSBSO, Alkyne-A-DSBSO) with a MS-cleavable trioxane-based cross-linker disclosed herein. In a particular embodiment, the program is a AlphaFold based program.

While it was shown that TSTO is highly effective for in-cell cross-linking at lower concentrations than bifunctional cross-linkers, the heterogeneity of TSTO cross-links appears to impact the number of unique PPIs obtained for proteome-wide analysis. However, it is noted that each interaction was identified with higher confidence as they were supported by an increased number of cross-links. To enhance protein coverage and increase cross-link identification for proteome-wide experiments, fractionation of cross-linked protein complexes can be coupled with peptide separation to improve analysis sensitivity and dynamic range. Use of polyclonal antibodies that can recognize MS-cleavable cross-linkers suggest that similar affinity purification strategies could be adapted to enrich crosslinked peptides using the trioxane-based MS cleavable cross-linkers of the disclosure, thereby increasing their MS detectability. Additionally, software programs can be developed that allow for identification of three cross-linked peptides from a single MS² spectrum. Due to the unique MS-cleavability of the trioxane-based MS cleavable cross-linkers disclosed herein, it is anticipated that MS²-based identification of the trioxane-based cross-links would be highly feasible compared to the identification of tri-links formed by conventional bifunctional cross-linkers. Given its unique ability to form trivalent cross-links and its robustness in cross-link identification, the trioxane-based MS cleavable cross-linkers of the disclosure represent a new class of cross-linkers which can provide additional spatial restraints to significantly enhance the understanding of protein modules and their organization in complex biological systems.

Kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more trioxane-based MS cleavable cross-linkers disclosed herein, optionally in a composition or in combination with another agent as disclosed herein. The container(s) typically are made to exclude or limit light exposure for the contents of the container. Such kits optionally comprise a MS-cleavable trioxane-based cross-linker disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a trioxane-based MS cleavable cross-linker described herein. Non-limiting examples of such materials include, but are not limited to, buffers, diluents; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific analytical application. The label can also indicate directions for use of the contents, such as in the methods described herein.

The disclosure further provides that the methods and compositions described herein can be further defined by the following aspects (aspects 1 to 39):

1. A trioxane-based mass spectrometry (MS)-cleavable cross-linker comprising:
a central trioxane group, which may be isotopically enriched with heavier isotopes selected from C¹³, and O¹⁸;
two or more MS-cleavable bonds;
two or more reactive cross-linking groups that can react with amino acids of peptides and/or proteins;
optionally, linker arms that connect the reactive cross-linking groups with the central trioxane group, wherein the linker arms may be isotopically enriched with heavier isotopes selected from H², C¹³, O¹⁸ and N¹⁵;
wherein the trioxane-based MS-cleavable cross-linker is configured to form a dimeric or trimeric cross-links with peptides and/or proteins.

2. The trioxane-based MS-cleavable cross-linker of aspect 1, wherein the two or more MS-cleavable bonds can be cleaved using collision-induced dissociation.

3. The trioxane-based MS-cleavable cross-linker of aspect 1 or aspect 2, wherein the two or more reactive cross-linking groups are located equal distant to the central trioxane group.

4. The trioxane-based MS-cleavable cross-linker of any one of aspects 1 to 3, wherein the two or more reactive cross-linking groups are selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative.

5. The trioxane-based MS-cleavable cross-linker of any one of aspects 1 to 4, wherein the central trioxane group comprises one or more of C¹³ and/or O¹⁸ atoms.

6. The trioxane-based MS-cleavable cross-linker of any one of aspects 1 to 5, wherein the trioxane-based mass spectrometry (MS)-cleavable cross-linker comprise 2 or more linker arms that connect the reactive cross-linking groups with the central trioxane group, wherein the linker arms may be isotopically enriched with heavier isotopes selected from H², C¹³, O¹⁸ and N¹⁵.

7. The trioxane-based MS-cleavable cross-linker of aspect 6, wherein one or more of the linker arms comprise one or more of H², C¹³, O¹⁸ and/or N¹⁵ atoms.

8. The trioxane-based MS-cleavable cross-linker of aspect 6 or aspect 7, wherein the trioxane-based mass spectrometry (MS)-cleavable cross-linker comprises 2 linker arms that connect the reactive cross-linking groups with the central trioxane group, and comprise a linker arm that connects an enrichable handle or a fluorophore to the central trioxane group.

9. The trioxane-based MS-cleavable cross-linker of aspect 8, wherein the enrichable handle is selected from a click chemistry linker, a phosphate, a fluorophore, biotin, an azide, an alkyne, and a phosphonic acid.

10. The trioxane-based MS-cleavable cross-linker of aspect 6 or aspect 7, wherein the trioxane-based mass spectrometry (MS)-cleavable cross-linker comprise 2 linker arms that connect the reactive cross-linking groups with the central trioxane group, and comprise a linker arm that connects an enrichable handle or a fluorophore to the central trioxane group.

11. The trioxane-based MS-cleavable cross-linker of aspect 6 or aspect 7, wherein the trioxane-based mass spectrometry (MS)-cleavable cross-linker comprises 3 linker arms that connect three reactive cross-linking groups with the central trioxane group.

12. The trioxane-based MS-cleavable cross-linker of aspect 11, wherein the three reactive cross-linking groups have different structures or have different atomic masses.

13. The trioxane-based MS-cleavable cross-linker of aspect 11, wherein two of the reactive cross-linking groups have the same structure, and one of the reactive cross-linking groups has a different structure or has a different atomic mass.

14. The trioxane-based MS-cleavable cross-linker of aspect 11, wherein all three of the reactive cross-linking groups have the same structure.

15. The trioxane-based MS-cleavable cross-linker of any one of aspects 1 to 14, wherein one of the reactive cross-linking groups comprises one or more of H², C¹³, O¹⁸ and/or N¹⁵ atoms.

16. The trioxane-based MS-cleavable cross-linker of any one of aspects 1 to 14, wherein two of the reactive cross-linking groups comprises one or more of H², C¹³, O¹⁸ and/or N¹⁵ atoms.

17. A trioxane-based mass spectrometry (MS)-cleavable cross-linker, wherein the trioxane-based MS-cleavable cross-linker has the structure of:

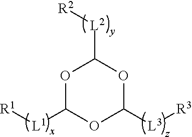

wherein,
L¹, L², and L³ are linker arms each individually selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide,

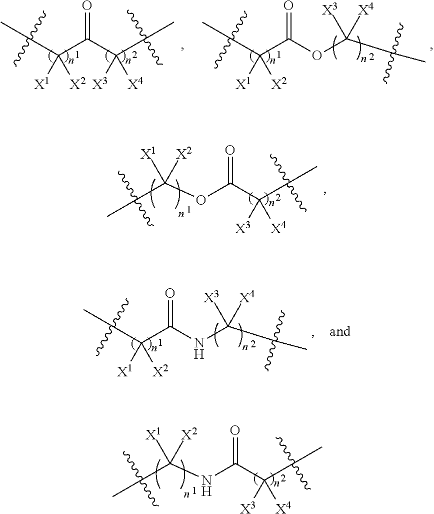

wherein L¹, L², and L³ may be isotopically enriched with heavier isotopes selected from H², C¹³, O¹⁸ and N¹⁵;
R¹, R², and R³ are individually selected from a reactive cross-linking group that can react with amino acids of peptides and/or proteins, an enrichable handle, and a fluorophore, wherein at least two of R¹ to R³ are reactive cross-linking groups;
X¹-X⁴ are each individually selected from FG, H, (C₁-C₆)alkyl, (C₁-C₆)alkenyl, (C₁-C₆)alkynyl, cyano, azide, hydroxyl, aldehyde, carboxyl, halo, amide, and amine, wherein each of the foregoing groups may be isotopically enriched with heavier isotopes selected from H², C¹³, O¹⁸ and/or N¹⁵;
x, y, and z are integers selected from 0 and 1; and
n¹ and n² are integers selected from 0, 1, 2, 3, 4, 5 and 6,
wherein the trioxane-based MS-cleavable cross-linker is configured to form dimeric or trimeric cross-links with amino acids of peptides and/or proteins.

18. The trioxane-based MS-cleavable cross-linker of aspect 17, wherein L¹ to L³ are individually selected from optionally substituted (C₁-C₆)alky,

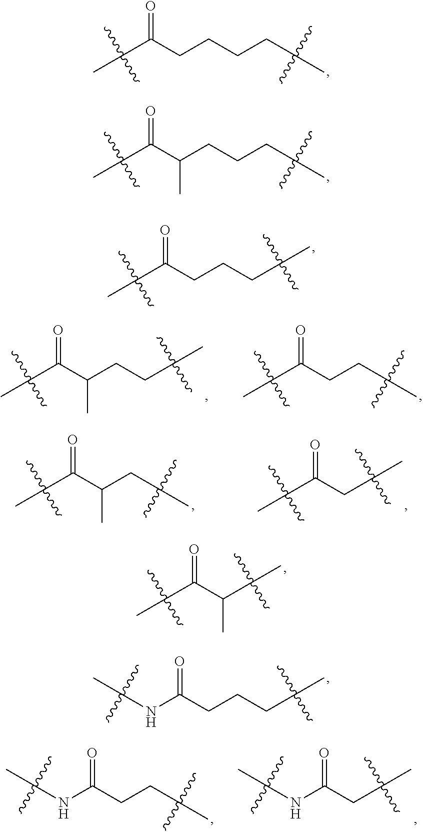

wherein each of the foregoing groups may be isotopically enriched with heavier isotopes selected from H², C¹³, O¹⁸ and N¹⁵.

19. The trioxane-based MS-cleavable cross-linker of aspect 17 or 18, wherein L¹ to L³ are

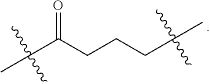 [SMILES: *CCCC(*)=O]

20. The trioxane-based MS-cleavable cross-linker of any one of aspects 17 to 19, wherein the reactive cross-linking group is selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative.

21. The trioxane-based MS-cleavable cross-linker of any one of aspects 17 to 20, wherein the reactive cross-linking group that can react with amino acids of peptides and/or proteins is selected from

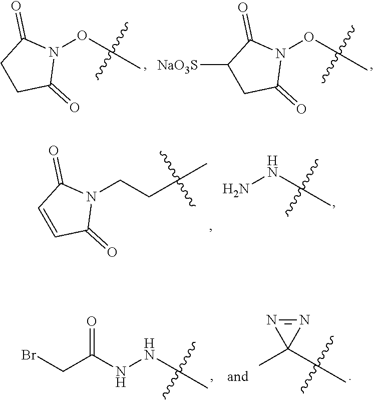

22. The trioxane-based MS-cleavable cross-linker of any one of aspects 17 to 21, wherein the enrichable handle is selected from a click chemistry linker, a phosphate, a fluorophore, biotin, an azide, an alkyne, and a phosphonic acid.

23. The trioxane-based MS-cleavable cross-linker of any one of aspects 17 to 22, wherein R¹, R², and R³ are reactive cross-linking groups, and wherein:
(i) at least two of Rt, R², and R³ have same structure, or
(ii) R¹, R², and R³ have same structure, or
(iii) R¹, R², and R³ have different structures.

24. The trioxane-based MS-cleavable cross-linker of any one of aspects 17 to 23, wherein the MS-cleavable trioxane-based cross-linker has the structure of:

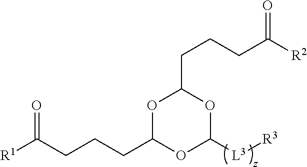

wherein,
L³ is a linker arm selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide,

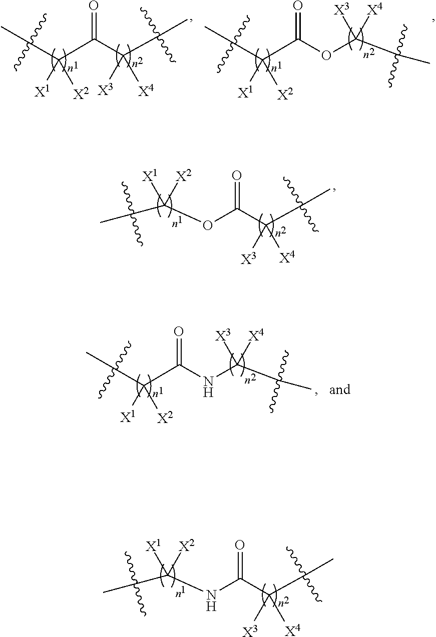

wherein L³ may be isotopically enriched with heavier isotopes selected from H², C¹³, O¹⁸ and N¹⁵;
R¹ and R² are

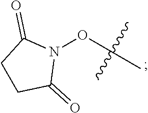 [SMILES: *ON1C(=O)CCC1=O]

R³ is selected from an enrichable handle, a fluorophore,

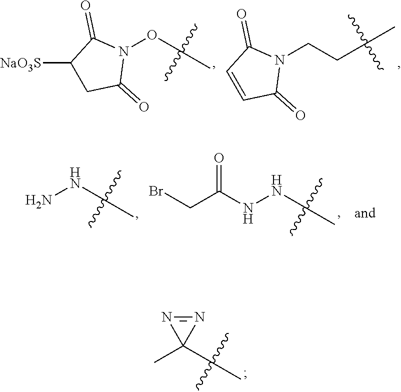

z is an integer selected from 0 and 1; and
n¹ and n² are integers selected from 0, 1, 2, 3, 4, 5 and 6.

25. The trioxane-based MS-cleavable cross-linker of any one of aspects 17 to 24, wherein the MS-cleavable trioxane-based cross-linker has the structure of:

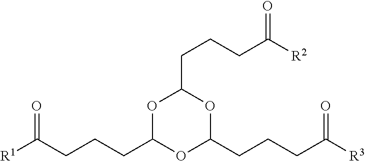

wherein, R¹-R³ are each individually selected from

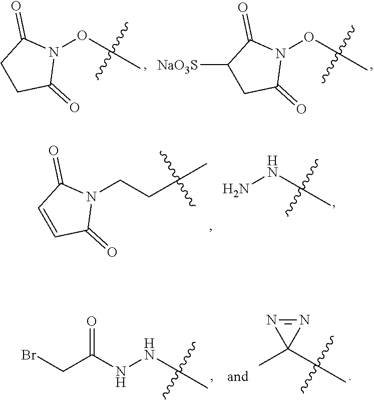

26. The trioxane-based MS-cleavable cross-linker of any one of aspects 17 to 25, wherein the MS-cleavable trioxane-based cross-linker is tris-succinimidyl trioxane (TSTO) having the structure of:

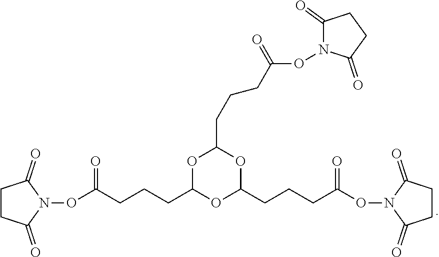 [SMILES: O=C(CCCC1OC(CCCC(=O)ON2C(=O)CCC2=O)OC(CCCC(=O)ON2C(=O)CCC2=O)O1)ON1C(=O)CCC1=O]

27. A method for mapping intra-protein interactions in a protein, inter-protein interactions in a protein complex, or any combination thereof, the method comprising:
contacting the protein and/or the protein complex comprising a plurality of cysteine moieties with the trioxane-based MS-cleavable cross-linker of any one of aspects 1 to 26 to form a cross-linked product;
digesting the cross-linked product to form a plurality of fragments, wherein a portion of the plurality of fragments comprises cross-linked peptide fragments; and
identifying and analyzing cross-linked peptide fragments using tandem mass spectrometry (MSⁿ) to map intra-protein interactions in the protein and/or inter-protein interactions in the protein complex.

28. The method of aspect 27, wherein the trioxane-based MS-cleavable cross-linker is used at a molar ratio of 1:10 to 10:1 to the protein and/or the protein complex.

29. The method of aspect 28, wherein the trioxane-based MS-cleavable cross-linker is used at a molar ratio of 1:5 to 5:1 to the protein and/or the protein complex.

30. The method of any one of aspects 27 to 28, wherein the cross-linked product was digested with one or more proteases.

31. The method of aspect 30, wherein the one or more proteases are serine proteases.

32. The method of any one of aspects 27 to 31, wherein a data-dependent MS³ acquisition method is used for identifying and analyzing the cross-linked peptide fragments.

33. The method of aspect 32, wherein the results of the data-dependent MS³ acquisition method is used with a computer program that preforms predictions of protein structure, particularly wherein the computer program is an artificial intelligence based program, more particularly, where the computer program is a AlphaFold based program.

34. A method for mapping global protein-protein interactions (PPIs) from a sample comprising a plurality of proteins;
contacting the sample comprising a plurality of proteins with the trioxane-based MS-cleavable cross-linker of any one of aspects 1 to 26 to form crosslinked proteins;
digesting the crosslinked proteins to form crosslinked protein fragments or peptides;
isolating fractions that are enriched with cross-linked protein fragments or peptides in the sample;
analyzing the fractions using tandem mass spectrometry (MSⁿ) and protein database searching to identify cross-linked protein fragments or peptides; and
mapping the identified cross-linked protein fragments or peptides to generate a global structural map of PPIs.

35. The method of aspect 34, wherein the sample is a tissue sample or a cellular sample.

36. The method of any one of aspect 34 or aspect 35, wherein the cross-linked product was digested with one or more proteases.

37. The method of aspect 36, wherein the one or more proteases are serine proteases.

38. The method of any one of aspects 34 to 37, wherein the fractions are isolated by using peptide size exclusion chromatography coupled with high pH reverse phase tip fractionation.

39. The method of any one of aspects 34 to 38, wherein a data-dependent MS³ acquisition method is used for identifying the cross-linked protein fragments or peptides.

40. The method of any one of aspects 34 to 39, wherein the identified cross-linked protein fragments or peptide are mapped using various programs or databases that profile protein to protein interactions.

41. The method of aspect 40, wherein the databases or programs utilize artificial intelligence, particularly, where the databases or programs is a AlphaFold based database or program.

The following examples are intended to illustrate but not limit the disclosure. While they are typical of those that might be used, other procedures known to those skilled in the art may alternatively be used.

### Examples

Materials and Reagents. All chemicals were purchased from Sigma-Aldrich, Acros Organics, Alfa Aesar, TCI, Advanced ChemTech, or Fisher and used without further purification unless otherwise noted. Zinc (II) chloride was flame dried under vacuum prior to use. Ethanol was purchased from Gold Shield. Solvents were of reagent grade and used as without further purification except as follows: N,N-dimethylformamide (DMF), dichloromethane (DCM), tetrahydrofuran (THF), and diethyl ether (ether) were degassed and then passed through anhydrous neutral alumina A-2 before use, according to the procedure described by Pangborn et al. (Organometallics 15(5):1518-1520 (1996)). Methanol was dried over activated 3 Å molecular sieves prior to use. Triethylamine was distilled over calcium hydride and stored over activated 3 Å molecular sieves prior to use. Diisopropylethylamine (DIPEA) was distilled over calcium hydride prior to use. Trifluoroacetic anhydride (TFAA) and trimethylsilyl triflate (TMS-OTf) were distilled prior to use. Reported reaction temperatures refer to the temperature of the heating medium. Reactions were performed in flame- or oven-dried glassware under an atmosphere of dry argon using standard Schlenk techniques unless otherwise noted. Room temperature (rt) refers to 25±3° C. Reactions were monitored by thin-layer chromatography (TLC) using EMD Chemicals Inc. silica gel 60 F256 plates. Flash chromatography was performed using Ultra Pure SiliaFlash P60, 230-400 mesh (40-63 μm) silica gel (SiO₂) following the general procedure by Still et al., (J. Org. Chem 43(14):2923-2925 (1978).

Animal experiments. Animal experiments were performed according to the protocol approved by the Institutional Animal Care and Use Committee (IACUC) of the University of California, Irvine. Heart tissue used in the experiments were obtained from adult (20-week-old) C57BL/6 wildtype mice. Mice were maintained at constant temperature (23° C.) and humidity and housed at 12-hour light/12-hour dark cycles and with free access to food and water.

Instrumentation. Proton NMR spectra measurements were acquired using either a Bruker DRX500 with a cryoprobe, Bruker GN500, or a Bruker AVANCE600 spectrometer, at 500 MHz, 500 MHz, and 600 MHz, respectively. Carbon NMR spectra were obtained on a Bruker DRX500 with a cryoprobe at 125 MHz. Proton NMR chemical shifts (δ) are reported in parts per million (ppm) and referenced to the residual solvent peak at 7.26 ppm for deuterated chloroform (CDCl₃) and 2.50 for deuterated dimethylsulfoxide (DMSO-d6). Carbon NMR chemical shifts (δ) are reported in parts per million (ppm) and referenced to the residual solvent peak at 77.16 ppm for deuterated chloroform and 39.52 for deuterated dimethylsulfoxide. All NMR spectra were processed using MestReNova (Mestrelab Research). NMR data are reported in the following manner: chemical shift, multiplicity, (s=singlet, d=doublet, t=triplet, q=quartet, quin=quintet, m=multiplet, br=broad, app=apparent), coupling constants (J) in hertz (Hz), and integration. High resolution mass spectrometry (HRMS) accurate mass experiments were performed by the University of California, Irvine mass spectrometry laboratory. Infrared (IR) spectroscopy data were acquired on a Shimadzu IRAffinity-1 Spectrophotometer with a MIRacle 10 single reflection ATR accessory. Melting points (mp) were acquired on a Mel-Temp melting point apparatus and are uncorrected. Tandem mass spectroscopy (MS/MS) analysis was performed on a Waters Quattro Premier XE mass spectrometer.

Synthesis of Trioxane CID-XLs. To test the fragmentation hypothesis of the trioxane core in MS/MS, model substrate 3-3 was synthesized (Scheme 1). In order to eventually synthesize a lysine-reactive cross-linker, an ester moiety was employed for two of the three arms of the trioxane. Acid catalyzed ring opening of delta-valerolactone in methanol provided methyl ester 3-1, which was converted to 3-2 via Swern oxidation in an 85% yield over two steps. Treatment of two equivalents of 3-2 and one equivalent of 3-phenylpropanal with 10 mol % zinc-(II) chloride afforded a nearly statistical distribution of trimerization products, as well as small amounts of starting materials. Trioxane 3-3 was isolated from the mixture by column chromatography in 30% yield.

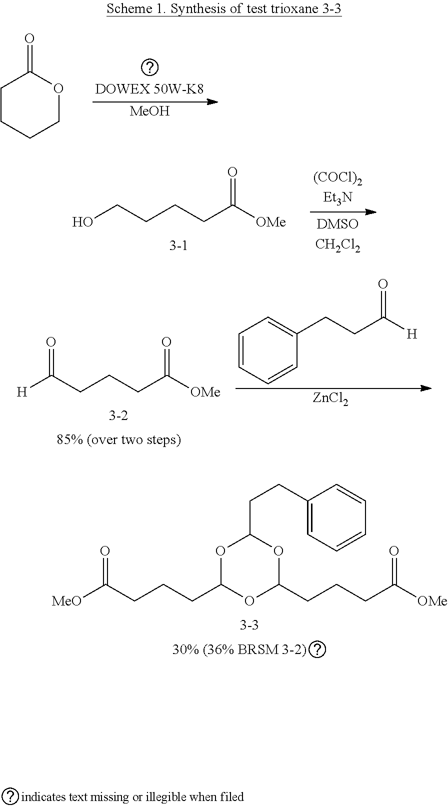

Trioxane 3-3 was subjected to MS/MS; however, despite utilizing a solvent of 20% formic acid in methanol, 3-3 was only detected as the sodium salt (M+Na⁺). The CID process is only effective for protonated molecules (M+H⁺), as an increase in stability is observed in the sodiated species, hindering an accurate approximation of the fragmentation energy. Therefore, the MS/MS data for 3-3 could not be compared to the MS/MS data of the diamidated lysine-reactive sulfoxide CID-XLs where the cross-linker was detected with a proton. Thus, the synthesis of the trioxane cross-linker 3-5 was carried out (Scheme 2). Saponification of 3-3 with lithium hydroxide afforded 3-4 in quantitative yield, and NHS-ester formation yielded the desired trioxane 3-5 in 63% yield after column chromatography. During cross-linking experiments, 3-5 was discovered to cleave before the peptide backbone, suggesting that it is an effective cross-linker.

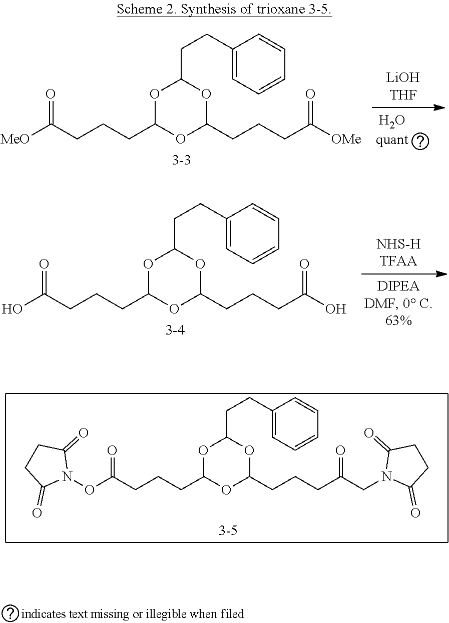 [SMILES: COC(=O)CCCC1OC(CCCC(=O)OC)OC(CCc2ccccc2)O1 ; O=C(O)CCCC1OC(CCCC(=O)O)OC(CCc2ccccc2)O1 ; O=C(CCCC1OC(CCCC(=O)ON2C(=O)CCC2=O)OC(CCc2ccccc2)O1)CN1C(=O)CCC1=O]

With knowledge that the trioxane moiety is susceptible in MS/MS, attempts were made to improve the overall yield of the trioxane cross-linker. As an alternative to the low yielding trimerization step to form 3-3, it was envisioned that trioxane 3-6 could be mono-functionalized. This would allow one ‘arm’ of the trioxane to be modified separately from the other arms. Initially, mono-hydrolysis was investigated (Scheme 3). Considering that concentration may be a significant factor, the reaction was run under dilute conditions in an attempt to diminish the competing di- and tri-hydrolysis reactions. Despite running the mono-hydrolysis at 0.01 M with a slow addition of potassium hydroxide, the maximum yield of the mono-hydrolysis product was 27%.

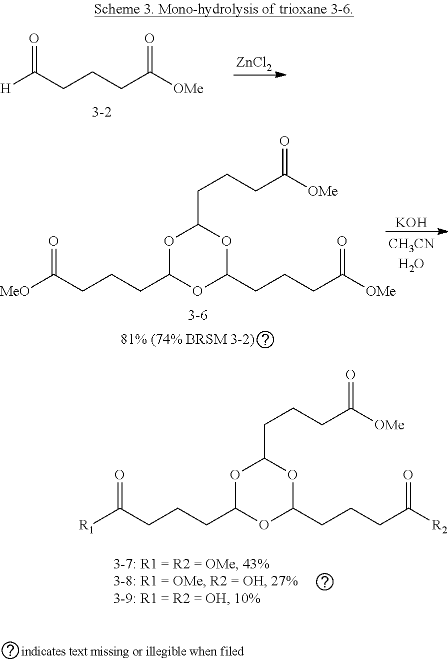

Alternatively, it was envisioned that mono-amidation could be applied to a symmetric triester system. Mono-amidation of symmetric diesters has been achieved in high yield with a number of different catalysts. Unfortunately, efforts to mono-amidate 23 using n-butylamine with various bases and catalysts did not afford any desired product (Scheme 4).

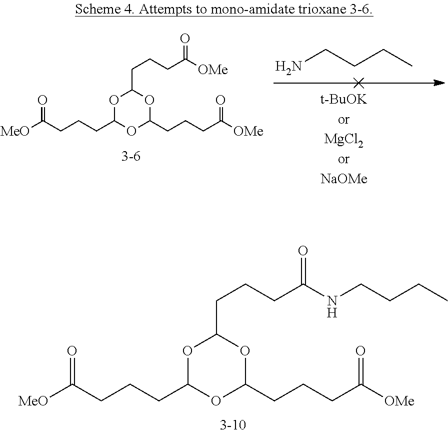

Mono-reduction of diesters with diisobutylaluminium hydride (DIBAL-H) has also been reported to proceed in high yield. After the reaction of 3-6 with DIBAL-H, trioxane aldehyde 3-11 was found to be inseparable from starting trioxane 3-6, so the crude reaction mixture was subjected to sodium borohydride reduction conditions to afford alcohol 3-12 (Scheme 5). Although trioxane 3-6 was recovered by column chromatography, the overall yield of desired trioxane alcohol 3-12 was low.

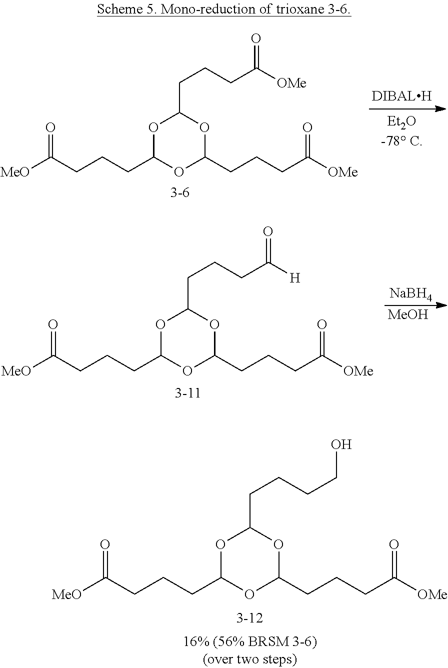 [SMILES: "MeOH" ; COC(=O)CCCC1OC(CCCC(=O)OC)OC(CCCC(=O)OC)O1 ; COC(=O)CCCC1OC(CCCC=O)OC(CCCC(=O)OC)O1 ; "NaBH4" ; COC(=O)CCCC1OC(CCCCO)OC(CCCC(=O)OC)O1]

A different attempt to synthesize the trioxane core was also investigated (Scheme 6). It was envisioned that a bicyclic compound such as 3-15 could be synthesized, after which the double bond would be oxidatively cleaved to afford a bi-functional cross-linker. Mono-oxidation of commercially available 1,5-cyclooctadiene afforded epoxide 3-13, which was followed by oxidative cleavage to yield alkene 3-14. Unfortunately, attempts to cyclize 3-14 with 3-2 were unsuccessful. Within five minutes of starting the reaction, the solution turned black, and upon workup followed by column chromatography only starting materials were recovered in a near quantitative amount. It was hypothesized that the zinc-(II) chloride was coordinating to the alkene in 3-14, impeding the reaction. Upon adding additional equivalents of the Lewis acid, the reaction still failed to afford 3-15, and still resulted in recovery of the starting materials in a near quantitative amount.

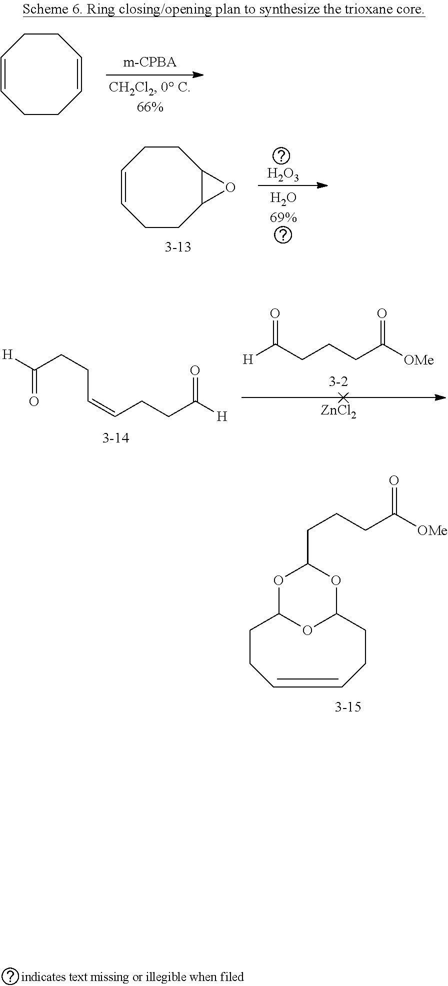

The unsuccessful mono-functionalization routes prompted the synthesis of a symmetrical trioxane (Scheme 7). Hydrolysis of symmetrical tri-ester 3-6 afforded tri-acid 3-16, which was subsequently hydrolyzed to yield symmetrical tri-NHS-ester 3-17.

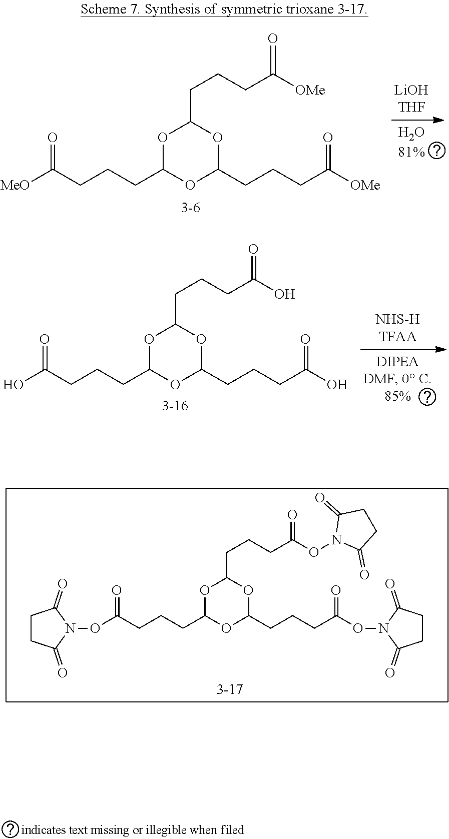 [SMILES: COC(=O)CCCC1OC(CCCC(=O)OC)OC(CCCC(=O)OC)O1 ; O=C(O)CCCC1OC(CCCC(=O)O)OC(CCCC(=O)O)O1 ; O=C(CCCC1OC(CCCC(=O)ON2C(=O)CCC2=O)OC(CCCC(=O)ON2C(=O)CCC2=O)O1)ON1C(=O)CCC1=O]

The trioxane functional group was found to cleave during MS/MS before the peptide backbone, suggesting that it is an effective scaffold to incorporate into CID-XLs for peptide sequencing. Trioxane CID-XL 3-5 was synthesized and worked well in initial biological testing. This inspired the synthesis of trioxane 3-17, which was further tested herein.

### Detailed Experimental Procedures for Producing Trioxane-Based Crosslinking Compounds.

N-hydroxysuccinimide Ester Formation from Diacids

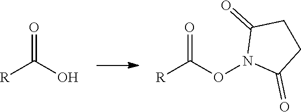

To a cooled (0° C.) mixture of the diacid (1 equiv), N-hydroxysuccinimide (4 equiv), and DIPEA (8 equiv) in DMF (0.2 M) was added TFAA (4 equiv) dropwise, slowly. The light orange solution was allowed to warm to rt and stir until determined complete by TLC, after which it was partitioned between ethyl acetate and hydrochloric acid (1 M). The layers were separated, and the acidic aqueous layer was extracted with ethyl acetate (2×). The organic layers were combined, washed with sodium bicarbonate solution (1 M, 3×), water (1×), and brine (1×). The organic layer was dried over anhydrous sodium sulfate, filtered, and concentrated in vacuo.

### Methyl 5-hydroxypentanoate (3-1)

¹H and ¹³C NMR spectra matched those previously reported for this compound in Hickmann et al. (J. Am. Chem. Soc. 133(34):13471-13480 (2011)).

### Methyl 5-oxopentanoate (3-2)

¹H and ¹³C NMR spectra matched those previously reported for this compound in Hickmann et al. (J Am. Chem. Soc. 133(34):13471-13480 (2011)).

### Dimethyl 4,4′-(6-phenethyl-1,3,5-trioxane-2,4-diyl)dibutanoate (3-3)

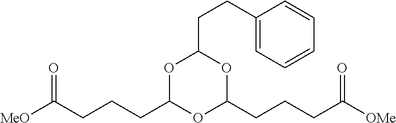 [SMILES: COC(=O)CCCC1OC(CCCC(=O)OC)OC(CCc2ccccc2)O1]

To zinc (II) chloride (0.009 g, 0.066 mmol) was added 3-2 (0.05 g, 0.38 mmol) and 3-phenylpropanal (0.025 g, 0.190 mmol) simultaneously. The resulting cloudy solution was let stir at rt for 16 h, after which it was diluted in ethyl acetate (100 mL). The reaction mixture was washed with water (3×30 mL), dried over anhydrous sodium sulfate, filtered, and concentrated in vacuo to a colorless oil. The crude reaction mixture was chromatographed (1:1 ethyl acetate:heaxanes) to afford 3-3 as a colorless oil (0.023 g, 30%): ¹H NMR (500 MHz, CDCl₃): δ 7.30-7.25 (m, 2H), 7.19-7.13 (m, 3H), 4.88-4.82 (m, 3H), 3.67 (s, 6H), 2.73 (t, J=7.7 Hz, 2H), 2.36 (t, J=7.0 Hz, 4H), 1.99 (q, J=6.6 Hz, 2H), 1.78-1.70 (m, 8H); ¹³C NMR (125 MHz, CDCl₃): δ 174.0, 141.4, 128.6, 128.5, 126.1, 101.1, 100.7, 51.7, 35.7, 33.8, 33.7, 29.7, 19.2; IR (thin film): 2848, 2359, 2340, 1735, 1435, 1361, 1128, 698 cm⁻¹; HRMS (ESI) m/z calcd for C₂₁H₃₀O₇Na [M+Na]⁺ 417.1889, found 417.1882.

### 4,4′-(6-Phenethyl-1,3,5-trioxane-2,4-diyl)dibutanoic acid (3-4)

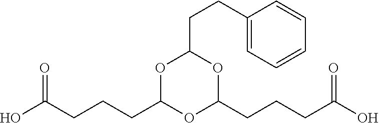 [SMILES: O=C(O)CCCC1OC(CCCC(=O)O)OC(CCc2ccccc2)O1]

To a stirred solution of 3-3 (0.03 g, 0.79 mmol) in THF (2 mL) and water (2 mL) was added LiOH (98%, 0.06 g, 2.50 mmol). The resulting cloudy solution was let stir for 12 h, after which it was acidified to pH 1 (monitored by pH paper) with hydrochloric acid (6 M). The aqueous layer was extracted with ethyl acetate (3×10 mL). The organic layers were combined, washed with water (3×15 mL), brine (1×15 mL), dried over anhydrous sodium sulfate, filtered, and concentrated in vacuo to afford 3-4 as a colorless oil (0.028 g, quant.): ¹H NMR (600 MHz, CDCl₃): δ 10.98 (br s, 2H), 7.30-7.25 (m, 2H), 7.19-7.13 (m, 3H), 4.87 (t, J=4.8 Hz, 2H), 4.84 (t, J=5.3 Hz, 1H), 2.79-2.68 (m, 2H), 2.41 (t, J=7.1 Hz, 4H), 2.05-1.95 (m, 2H), 1.86-1.68 (m, 8H); ¹³C NMR (125 MHz, CDCl₃): δ 180.0, 141.4, 128.58, 128.57, 128.54, 126.1, 101.0, 100.7, 35.7, 33.8, 33.5, 29.7, 18.9; IR (thin film): 3126, 2930, 1709, 1128, 1070 cm⁻¹; HRMS (ESI) m/z calcd for C₁₉H₂₅O₇ [M−H]⁻ 365.1600, found 365.1607.

### Bis(2,5-dioxopyrrolidin-1-yl) 4,4′-(6-phenethyl-1,3,5-trioxane-2,4-diyl)dibutanoate (3-5)

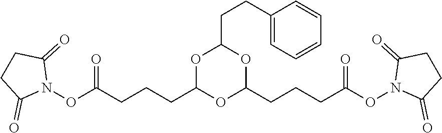 [SMILES: O=C(CCCC1OC(CCCC(=O)ON2C(=O)CCC2=O)OC(CCc2ccccc2)O1)ON1C(=O)CCC1=O]

Diacid 3-4 (0.028 g, 0.076 mmol) was subjected to general procedure 3.1 to afford a colorless oil. The crude product was subjected to column chromatography (3:1 ethyl acetate:hexanes) to afford 3-5 as a colorless oil (0.027 g, 63%): ¹H NMR (500 MHz, CDCl₃): δ 7.28-7.25 (m, 2H), 7.22-7.15 (m, 3H), 4.90 (t, J=4.8 Hz, 2H), 4.84 (t, J=5.2 Hz, 1H), 2.83 (br s, 8H), 2.77-2.71 (m, 2H), 2.68 (t, J=7.4 Hz, 4H), 2.04-1.96 (m, 2H), 1.89-1.82 (m, 4H), 1.81-1.70 (m, 4H). ¹³C NMR (125 MHz, CDCl₃): δ 169.3, 168.5, 141.4, 128.57, 128.52, 126.0, 100.65, 100.63, 35.7, 33.1, 30.7, 29.6, 25.7, 18.7; IR (thin film): 2931, 1811, 1736, 1363, 1203, 1066 cm⁻¹; HRMS (ESI) m/z calcd for C₁₄H₁₆N₂O₉SNa [M+Na]⁺ 583.1904, found 583.1921.

### Trimethyl 4,4′,4″-(1,3,5-trioxane-2,4,6-triyl)tributanoate (3-6)

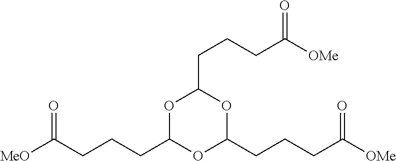 [SMILES: COC(=O)CCCC1OC(CCCC(=O)OC)OC(CCCC(=O)OC)O1]

To zinc (II) chloride (0.077 g, 0.560 mmol) was added 3-2 (0.49 g, 3.80 mmol). The resulting cloudy solution was let stir at rt for 12 h, after which it was diluted in ethyl acetate (60 mL). The reaction mixture was washed with water (3×30 mL), dried over anhydrous sodium sulfate, filtered, and concentrated in vacuo to a colorless oil. The crude reaction mixture was subjected to column chromatography (1:3.5:14 acetonitrile:ethyl acetate:heaxanes) to afford 3-6 as a colorless oil (0.300 g, 61%): ¹H NMR (500 MHz, CDCl₃): δ 4.86 (t, J=4.9 Hz, 3H), 3.67 (s, 9H), 2.35 (t, J=7.3 Hz, 6H), 1.83-1.64 (m, 12H); ¹³C NMR (125 MHz, CDCl₃): δ 173.9, 101.1, 51.7, 33.8, 33.7, 19.1; IR (thin film): 2955, 2863, 1734, 1710, 1436, 1170, 1129 cm⁻¹; HRMS (ESI) m/z calcd for C₁₈H₃₀O9Na [M+Na]⁺ 413.1787, found 413.1782.

### Attempted Mono-Hydrolysis of trimethyl 4,4′,4″-(1,3,5-trioxane-2,4,6-triyl)tributanoate (3-6)

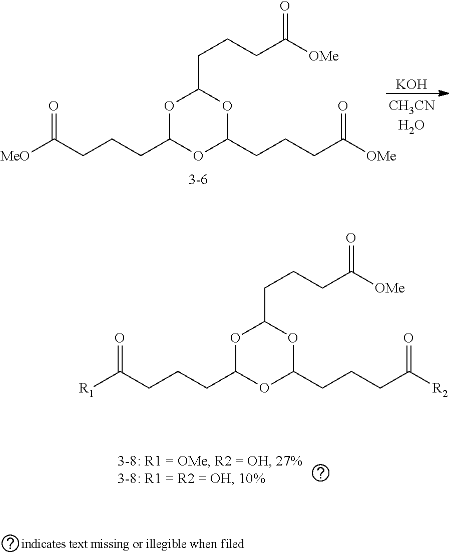

Triester 3-6 (0.053 g, 0.14 mmol) was dissolved in acetonitrile (0.25 mL), after which water (10 mL) was added. To the cooled (0° C.) solution was added potassium hydroxide dropwise (0.05 M solution, 2.7 mL) via syringe pump over 1 h. The resulting colorless solution was let stir for 1 h, after which the cooled (0° C.) crude reaction was carefully acidified to a pH of 1 (monitored by pH paper) with sulfuric acid (18 M). The acidified aqueous layer was extracted with ethyl acetate (4×10 mL). The organic layers were combined, washed with water (1×20 mL), dried over anhydrous sodium sulfate, filtered, and concentrated in vacuo to a colorless oil. The crude product was subjected to column chromatography (1:6:12 acetonitrile:ethyl acetate:heaxanes, with 1% glacial acetic acid) to afford 3-8 (0.014 g, 27%) as a colorless oil and 3-9 (0.005 g, 10%) as a colorless oil.

### 4-(4,6-Bis(4-methoxy-4-oxobutyl)-1,3,5-trioxan-2-yl)butanoic acid (3-8)

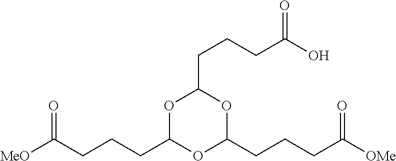 [SMILES: COC(=O)CCCC1OC(CCCC(=O)O)OC(CCCC(=O)OC)O1]

¹H NMR (500 MHz, CDCl₃): δ 9.77 (br s, 2H), 4.88-4.85 (m, 3H), 3.66 (s, 6H), 2.40 (t, J=7.0 Hz, 2H), 2.35 (t, J=7.1 Hz, 4H), 1.84-1.60 (m, 12H); 13C NMR (125 MHz, CDCl₃): δ 179.0, 174.0, 101.08, 101.05, 51.72, 51.70, 33.8, 33.6, 19.1, 18.9, 18.8; IR (thin film): 2955, 2359, 1733, 1436, 1167, 1127 cm-1; HRMS (ESI) m/z calcd for C₁₇H₂₈O₉Na [M+Na]⁺ 399.1631, found 399.1645.

### 4,4′-(6-(4-Methoxy-4-oxobutyl)-1,3,5-trioxane-2,4-diyl)dibutanoic acid (3-9)

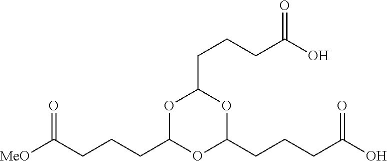 [SMILES: COC(=O)CCCC1OC(CCCC(=O)O)OC(CCCC(=O)O)O1]

¹H NMR (500 MHz, CDCl₃): δ 10.07 (br s, 2H), 5.00-4.79 (m, 3H), 3.67 (s, 3H), 2.40 (t, J=6.8 Hz, 4H), 2.35 (t, J=7.2 Hz, 2H), 1.91-1.57 (m, 12H); 13C NMR (125 MHz, CDCl₃): δ 179.7, 174.1, 101.1, 101.0, 53.6, 51.7, 33.8, 33.6, 33.4, 19.1, 19.0; IR (thin film): 2954, 2861, 2363, 1734, 1436, 1167 cm-1; HRMS (ESI) m/z calcd for C₁₆H₂₆O₉Na [M+Na]⁺ 385.1475, found 385.1479.

### Dimethyl 4,4′-(6-(4-hydroxybutyl)-1,3,5-trioxane-2,4-diyl)dibutanoate (3-12)

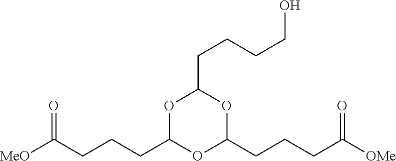 [SMILES: COC(=O)CCCC1OC(CCCCO)OC(CCCC(=O)OC)O1]

To a cooled (−78° C.) solution of 3-6 (0.113 g, 0.289 mmol) in ether (5 mL) was added diisobutylaluminium hydride (1M solution in hexanes, 0.70 mL) slowly, dropwise. The reaction was let stir 45 min at −78° C., after which water (0.5 mL) was added. The reaction was let warm to rt, after which it was dried over anhydrous sodium sulfate, filtered, and concentrated in vacuo to a colorless oil. The oil was dissolved in methanol (2 mL), after which THF (0.5 mL) and sodium borohydride (0.04 g, 1.06 mmol) were added. The colorless solution was let stir 12 h, after which it was partitioned between hydrochloric acid (0.25M, 60 mL) and ethyl acetate (60 mL). The layers were separated, and the aqueous layer was extracted with ethyl acetate (60 mL). The combined organic layers were washed with water (1×40 mL), dried over anhydrous sodium sulfate, filtered, and concentrated in vacuo to a colorless oil. The crude product was subjected to column chromatography (3:2 ethyl acetate:heaxanes) to afford 3-12 as a colorless oil (0.017 g, 16% over two steps): ¹H NMR (500 MHz, CDCl₃): δ 4.87-4.84 (m, 3H), 3.67-3.61 (m, 8H), 2.34 (t, J=7.1 Hz, 4H), 1.82-1.64 (m, 8H), 1.59-1.55 (m, 4H), 1.53-1.41 (m, 3H); 13C NMR (125 MHz, CDCl₃): δ 174.0, 101.4, 101.1, 62.8, 51.7, 34.1, 33.8, 33.7, 32.5, 19.8, 19.2; IR (thin film): 2954, 2358, 1733, 1436, 1249, 1160 cm⁻¹; HRMS (ESI) m/z calcd for C₁₇H₃₀O₈Na [M+Na]⁺ 385.1838, found 385.1843.

### 4,4′,4″-(1,3,5-Trioxane-2,4,6-triyl)tributanoic acid (3-16)

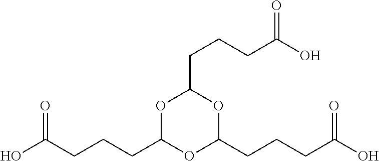 [SMILES: O=C(O)CCCC1OC(CCCC(=O)O)OC(CCCC(=O)O)O1]

To triester 3-6 (0.460 g, 1.18 mmol) in a solution of THF (30 mL) was added a solution of lithium hydroxide monohydrate (1.6 g, 38 mmol) in an equal volume of H₂O (30 mL). The cloudy reaction was let stir vigorously at rt overnight. In the morning, the crude reaction was cooled (0° C.) and then carefully acidified to a pH of 1 (monitored by pH paper) with sulfuric acid (18 M). The acidified aqueous layer was extracted with ethyl acetate (3×125 mL). The organic layers were combined, washed with water (50 mL), dried over anhydrous sodium sulfate, filtered, and concentrated in vacuo to give an off-white solid. Trituration in hexanes (10 mL) afforded 3-16 as a white solid (0.375 g, 91%): mp 129-130° C.; ¹H NMR (500 MHz, CDCl₃) δ 12.02 (s, 3H), 4.95 (t, J=3.9 Hz, 3H), 2.23 (t, J=6.4 Hz, 6H), 1.60-1.50 (m, 12H); 13C NMR (125 MHz, CDCl₃) δ 174.3, 100.2, 33.2, 33.2, 18.7; IR (thin film): 3394, 2960, 1703, 1407, 1293, 1126 cm⁻¹; HRMS (ESI) m/z calcd for C₁₅H₂₄O₉Na [M+Na]⁺ 371.1318, found 371.1327.

### Tris(2,5-dioxopyrrolidin-1-yl) 4,4′,4″-(1,3,5-trioxane-2,4,6-triyl)tributanoate (3-17)

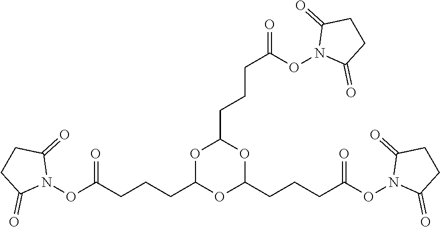 [SMILES: O=C(CCCC1OC(CCCC(=O)ON2C(=O)CCC2=O)OC(CCCC(=O)ON2C(=O)CCC2=O)O1)ON1C(=O)CCC1=O]

Triacid 3-16 (1.5 g, 4.3 mmol) was subjected to general procedure 3.1 to give a dark orange oil. Many triturations and recrystallizations were attempted with various mixtures of hexanes and ethyl acetate, but no pure product was obtained. Thus, column chromatography (4:1 ethyl acetate:heaxanes) afforded 3-17 as alight orange solid (2.53 g, 85%): mp 115-117° C.; ¹H NMR (600 MHz, CDCl₃) δ 4.92 (t, J=4.7 Hz, 3H), 2.81 (s, 12H), 2.66 (t, J=7.3 Hz, 6H), 1.94-1.82 (m, 6H), 1.81-1.74 (m, 6H); ¹³C NMR (125 MHz, CDCl₃) δ 169.3, 168.5, 100.6, 33.0, 30.7, 25.7, 18.7; IR (thin film): 2946, 1779, 1729, 1360, 1200 cm⁻¹; HRMS (ESI) m/z calcd for C₂₇H₃₃N₃O₁₅Na [M+Na]⁺ 662.1809, found 662.1795.

TSTO cross-linking of synthetic peptide Ac-SR8. Synthetic peptide Ac-SR8 was cross-linked at a concentration of 1 mM with 1 mM TSTO in anhydrous DMSO or pH 7.4 phosphate buffered saline. The reaction was carried out at RT with gentle vortexing, with aliquots of the original reaction volume being removed and quenched with ammonium bicarbonate at 5, 15, 30, and 60 min. After dilution by 1:500, the resulting cross-linked peptide mixtures were directly injected for MS analyses.

TSTO cross-linking of BSA. BSA was cross-linked at 20 mM with 1 mM TSTO in pH 7.4 phosphate buffered saline. The reaction was carried out for 1 h at RT, followed by quenching with ammonium bicarbonate. The protein was then reduced with tris(2-carboxyethyl)phosphine (TCEP) for 30 min at RT and alkylated with iodoacetamide in the dark at RT for 30 min. Cross-linked proteins were then digested in 8M urea buffer using LysC for 4 h at 37° C., followed by trypsin digestion at 37° C. overnight after diluting urea concentration to <1.5 M. The resulting peptide mixtures were extracted and desalted using C18 tips (Agilent) prior to MS analyses.

Affinity purification and TSTO cross-linking of human 26S proteasomes. A stable 293^{HBTH-Rpn1} cell line was first grown to confluency. After native cell lysis, human 26S proteasomes were purified from the clarified lysate by binding to streptavidin-sepharose resin. Bead-bound proteasomes were cross-linked on-bead in PBS buffer (pH 7.5) with 0.75 mM TSTO for 1 h at room temperature. After quenching the cross-linking reaction using ammonium bicarbonate, the proteins were reduced with TCEP for 30 min at RT and alkylated with iodoacetamide in the dark at RT for 30 min. Cross-linked proteins were then digested in 8M urea buffer using LysC for 4 h at 37° C., followed by trypsin digestion at 37° C. overnight after diluting urea concentration to <1.5 M. The resulting peptide mixtures were extracted and desalted using C18 tips (Agilent) prior to MS analyses.

Optimization of in vivo TSTO cross-linking by immunoblot analysis. A stable 293^{HBTH-CSN2} cell line was first grown to confluency, washed with PBS, and then gently pelleted. To determine the optimal cross-linking conditions, intact cells were cross-linked at various cross-linking concentrations ranging from 0.5 to 3 mM, at room temperature or 37° C. Clarified lysates from each condition were separated by SDS-PAGE and transferred onto a PVDF membrane and stained using amido black. After rinsing off the dye and blocking using 5% milk in TBST, the membrane-bound proteins were incubated with streptavidin-HRP to monitor the oligomerization of HBTH-CSN2 in response to cross-linking conditions. Based on these results, in vivo cross-linking of intact HEK293 cells was performed at 1 mM.

In vivo TSTO cross-linking of human cells. Intact 293^{HBTH-CSN2} cells were cross-linked using 1 mM TSTO in PBS buffer (pH 7.4) for 1 h with rotation at room temperature. Afterwards, the cross-linking reaction was quenched using excess ammonium bicarbonate (50 mM) for 10 min. Cells were spun down, washed again with PBS, and needle-lysed on ice in denaturing buffer (8 M urea, 50 mM Tris-HCl pH 7.5). Lysate was clarified by centrifugation at 21,000×g for 15 min in 4° C. and the resulting supernatant was transferred to EMD Millipore 30,000 NMWL Microcon centrifugal tubes for FASP digestion similarly as described in Wisniewski et al. (Nat Methods 6:359-362 (2009)). Briefly, proteins atop the filter were reduced using 10 mM TCEP for 20 min at RT, alkylated using 20 mM iodoacetamide in the dark at RT for 20 min, and then digested in 8 M urea buffer using LysC for 4 h at 37° C. followed by trypsin digestion at 37° C. overnight after urea dilution to 1.5 M. The resulting cross-linked peptide mixtures were then spun through the filter and desalted using Waters C18 Sep-Pak cartridges.

In vivo TSTO cross-linking of mouse heart tissue. Freshly excised mouse hearts were sliced into 1-2 mm cubes and washed in cold PBS to remove blood. The cubed cardiac tissue was incubated in 3 mL of 1 mM TSTO in PBS for 1 h at RT with rotation. The cross-linking reaction was then quenched using 20 mM ammonium bicarbonate for an additional 15 min rotation at RT. The tubes were then gently centrifuged and the tissues were washed using cold PBS twice. The tissues were then flash frozen and manually cryopulverized before lysis using a BioRuptor in denaturing buffer (8 M urea, 50 mM Tris-HCl pH 7.5). 5 cycles consisting of 30 sec sonication followed by 30 sec rest were used to lyse the cryopulverized tissue. Lysate was clarified by centrifugation at 21,000×g for 15 min in 4° C. and the resulting supernatant was transferred to EMD Millipore 30,000 NMWL Microcon centrifugal tubes for FASP digestion as described above. Following digestion with LysC and trypsin, the resulting cross-linked peptide mixtures were then spun through the filter and desalted using Waters C18 Sep-Pak cartridges.

SEC-HpHt enrichment of cross-linked peptides. Peptide separation by SEC was performed similarly as described in Leitner et al. (Mol Cell Proteomics 11:M111.014126 (2012)). Briefly, dried peptides were reconstituted in SEC mobile phase (0.1% formic acid and 30% ACN) and separated on a Superdex Peptide PC 3.2/30 column (300×3.2 mm) at a flow rate of 50 μL/min, monitored at 215, 254 and 280 nm UV absorbance. Two-minute fractions were collected, and only fractions 24 and 26 containing the most cross-linked peptides were collected. SEC-separated fractions were then vacuum dried and resuspended in ammonium water (pH 10) For high-pH separation, HpHt tips were prepared as described in Jiao et al. (Anal Chem 94:4236-4242 (2022)). Pipette tips (200 μL) were first blocked with a layer of C8 membrane (Empore 3M), then filled with 5 mg of C18 solid phase (3 μm, Durashell, Phenomenex). The tips were then washed sequentially with 90 μL of methanol, 90 μL of ACN and 90 μL of ammonia water (pH 10). After loading onto the tip, the peptides were washed once with 90 uL ammonia water and eluted using increasing percentage of ACN in ammonia water (6%, 9%, 12%, 15%, 18%, 21%, 25%, 30%, 35%, and 50%). The 25%, 30%, 35% and 50% fractions were then combined with 6%, 9%, 12% and 21% fractions, respectively. The final SEC-HpHt fractions were vacuum dried and stored at −80° C. before MS analysis.

LC-MSⁿ analysis. LC-MSⁿ analysis of cross-linked peptides was performed using an UltiMate 3000 UPLC (Thermo Fisher Scientific) liquid chromatograph coupled on-line to an Orbitrap Fusion Lumos mass spectrometer (Thermo Fisher Scientific). Peptides were separated by reverse-phase on a 50 cm×75 μm I.D. Acclaim® PepMap RSLC column using gradients of 4% to 25% acetonitrile at a flow rate of 300 nL/min (solvent A: 100% H₂O, 0.1% formic acid; solvent B: 100% acetonitrile, 0.1% formic acid) prior to MSⁿ analysis. For each MSⁿ acquisition, duty cycles consisted of one full Fourier transform scan mass spectrum (375-1500 m/z, resolution of 60,000 at m/z 400) followed by data-dependent MS² and MS³ acquired at top speed in the Orbitrap and linear ion trap, respectively. Ions detected in MS¹ with 4+ or greater charge were selected and subjected to CID fragmentation (NCE 23%) in MS² and resulting ions were detected in the Orbitrap (resolution 30,000). Ions observed in MS² spectra with charge 2+ or greater were selected and fragmented in MS³ using CID (NCE 35%) and detected in the linear ion trap in ‘Rapid’ mode. Ions selected for MS³ were either based on abundance (top 4 or 5 most intense ions in MS²) or targeted based on doublets with mass difference pairs (Δ=18.02 Da) corresponding to cross-linker remnant moiety water loss. For 26S proteasome cross-links, each acquisition was 200 min and ion selection for MS³ was based on top intensity. For SEC-separated F24 fractions, the entire HpHt fraction was injected; for F26 fractions half of the HpHt fraction was injected for each LC-MSⁿ analysis. Each acquisition was 120 or 150 min, and both top intensity and mass difference-targeted methods were used for selecting ions for MS³ analysis.

Identification of TSTO Cross-links by MSⁿ. Spectrometric data were extracted from .raw files using PAVA. Extracted MS³ spectra were subjected to protein database searching via Batch-Tag within a developmental version of Protein Prospector (v. 6.3.5, University of California, San Francisco) against a randomly concatenated SwissProt database consisting of 20,418 human proteins and their corresponding decoys. Mass tolerances for parent ions and fragment ions were set as ±15 ppm and 0.6 Da, respectively. Trypsin was set as the enzyme with three maximum missed cleavages allowed. Cysteine carbamidomethylation was selected as a constant modification, while protein N-terminal acetylation, methionine oxidation, and N-terminal conversion of glutamine to pyroglutamic acid were selected as variable modifications. Two additional defined variable modifications on uncleaved lysines and free protein N-termini were selected: AR (C₅H₆O₂, 98.04 Da) and AR* (C₅H₄O, 80.03 Da), corresponding to remnant moieties for cleaved TSTO. MSⁿ data (monoisotopic masses and charges of parent ions and corresponding fragment ions and MS³ database search results were integrated via in-house software xl-Tools to automatically generate, summarize and validate identified cross-linked peptide pairs. Experimental FDR calculated using a target-decoy approach was determined to be 0.04% at the cross-link level. Using a minimum peptide length of 5 residues for individual cross-linked peptides, separation of inter/intra FDR calculation resulted in experimental FDRs of 0.2% and 0.0%, respectively.

Integrative Modeling using TSTO data. Integrative modeling was used to demonstrate the effectiveness of the TSTO cross-linker in structural modeling by computing the structure of the 26S human proteasome base subcomplex (subunits Rpt1-Rpt6, and Rpn2) based on data from trifunctional and bifunctional cross-linkers. Our protocol proceeded through the standard four stages:

(1) Gathering information: Input information consisted of AlphaFold2-predicted structures for the subunits (Rpt1-Rpt6 and Rpn2), 642 TSTO cross-links (XLs) identified in this study, 281 DSSO cross-links reported by Yu et al. (Anal Chem 94:4390-4398 (2022)), and synthetically generated cross-linking datasets. Synthetic cross-linking datasets were generated based on the cryo-EM structure of the human 26S proteasome (PDB ID: 5GJR). Cross-linking probabilities were assigned to lysine residue pairs with Cα-Cα distances below 30 Å using a skewed normal distribution, with parameters (location: 8.0, scale: 14.8, skewness: 7.6) derived from fitting the experimental distance distribution of trivalent cross-links. Monte Carlo sampling, guided by the assigned probabilities and the Metropolis acceptance criterion, was used to select residue pairs and triplets for constructing the synthetic datasets.

(2) Representing the subunits and translating data into spatial restraints: Relying on the AlphaFold2 predictions of the proteasome base subcomplex, each subunit was represented as a rigid body using a 1-residue-per-bead representation. Spatial restraints for cross-linked residues were applied as upper bounds on the distances between residue pairs. TSTO trivalent cross-links were represented by a set of three distance restraints between all residue pairs ensuring that distances spanned by all residue pairs are simultaneously within the cross-linker distance threshold. TSTO and DSSO bivalent cross-links were modelled using a single upper-bound distance restraint. Furthermore, excluded volume and sequence connectivity restraints were imposed on all components.

(3) Sampling: Structural models were computed using Replica Exchange Gibbs sampling, based on the Metropolis Monte Carlo (MC) algorithm. Each MC step consisted of a series of random transformations (i.e., rotations and/or translations) of the rigid bodies.

(4) Analysis and validation of the structural models of the 26s proteasome followed the five steps presented in Jumper et al. (Nature 596:583-589 (2021)), Saltzberg et al. (Protein Sci 30:250-261 (2021)), and Viswanath et al. (Biophysical Journal 133:2344-2353 (2017)).

To benchmark the four-stage protocol described here and assess the utility of the TSTO cross-linker for integrative structure modeling, we computed the distribution of the accuracy for the structural ensembles obtained by integrative modeling. The accuracy is defined as the mean of Cu RMSD between the EM structure and each of the structures in the ensemble. Additionally, we computed the precision of each ensemble of models; the precision is defined as the average RMSD between all solutions in the ensemble.

The integrative structure modeling protocol [i.e., stages (ii), (iii), and (iv)] was scripted using the Python Modeling Interface (PMI) package, a library for modeling macromolecular complexes based on our open-source Integrative Modeling Platform (IMP) package version 2.20. All input files, scripts, and output files are available at Hypertext Transfer Protocol [//github.com/salilab/pmi].

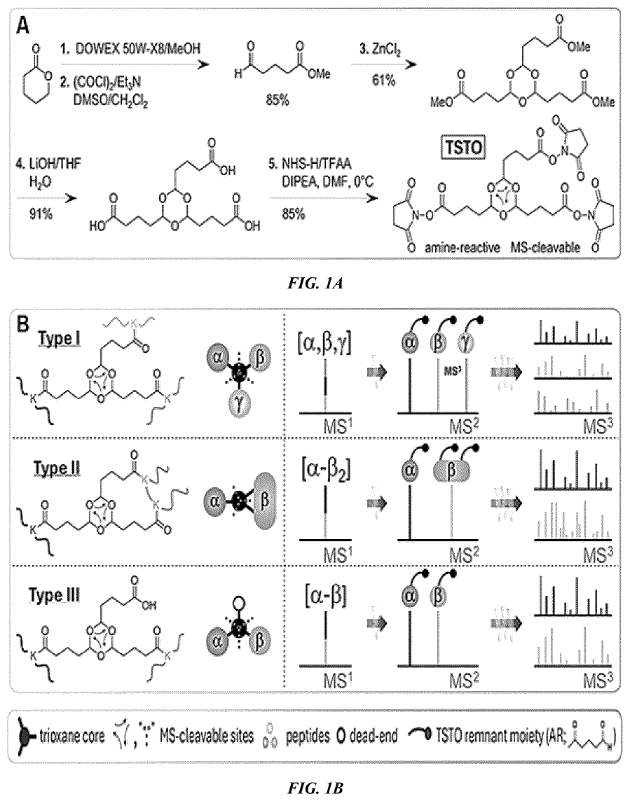

Developing a Trioxane-based MS-cleavable Homotrifunctional Cross-linker TSTO. In order to capture trimeric interactions, we designed a trioxane-based cross-linker TSTO was designed and accomplished through five steps (see FIG. 1A). As shown, TSTO carries a unique symmetrical structure comprising three NHS esters connected via a central trioxane group (see FIG. 1A), permitting concurrent cross-linking between three lysine residues to form a trivalent cross-link among three individual peptides (aka tripeptide tri-link, [α,β,γ]) (see FIG. 1B, Type I). In comparison to a traditional cross-link between two individual peptides, accurate identification of a tri-link would be much more challenging due to further expansion of database search space (n³). Therefore, the design of the central trioxane is important as it carries three equal MS-cleavable bonds that are weaker than peptide bonds and can be cleaved in parallel using CID to simultaneously release all three cross-linker arms in a single step. This leads to physical separation of the three cross-linked peptide constituents, yielding three fragment ions during MS² analysis that can be subjected to MS³ for sequencing (see FIG. 1B, Type I). As shown, trioxane cleavage results in an identical and defined aldehyde remnant (AR) on each peptide constituent, allowing for their unambiguous identification. In addition, this minimizes the total number of MS² fragments, simplifying ion selection for subsequent MS³ analysis. In addition to tripeptide TSTO cross-links, tri-links can be formed between two peptides, linking one lysine in one peptide (α) and two lysines in another peptide (β) (aka dipeptide ti-link, [α-β₂]) (see FIG. 1B, Type II). For dipeptide tri-links, two fragment ions would be observed in MS², one corresponding to a peptide carrying a single AR-modified lysine and the other representing a peptide carrying two AR-modified lysines. Finally, TSTO cross-linking can yield traditional cross-links in which two lysines from two different peptides are cross-linked while the third NHS ester of TSTO is hydrolyzed (aka, dipeptide bi-link, [α-β]) (see FIG. 1B, Type III). MS² analysis of a dipeptide bi-link would yield two fragment ions corresponding to peptides each carrying a single AR-modified lysine, while the hydrolyzed arm is released as a neutral loss. In addition to TSTO inter-links formed by two or three peptides as described above, monomeric cross-linked species containing a single peptide including ‘intra-links’ and ‘dead-ends’ can occur due to the hydrolysis of one or two NHS esters of TSTO. Notably, TSTO inter-linked peptides are the most structurally informative products for PPI mapping, and thus the focus of the analysis. Owing to their unique MS-cleavability, TSTO cross-linked peptides can be identified using the same LC-MSⁿ workflow for sulfoxide-containing MS-cleavable cross-linkers presented in Yu et al. (Anal Chem 90:144-165 (2018)), Yu et al. (Curr Opin Chem Biol 76:102357 (2023)), and Kao et al. (Mol Cell Proteomics 10:M110.002212 (2011)).

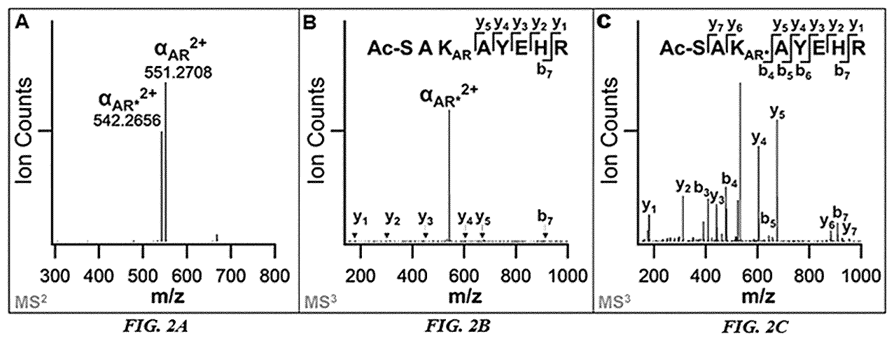

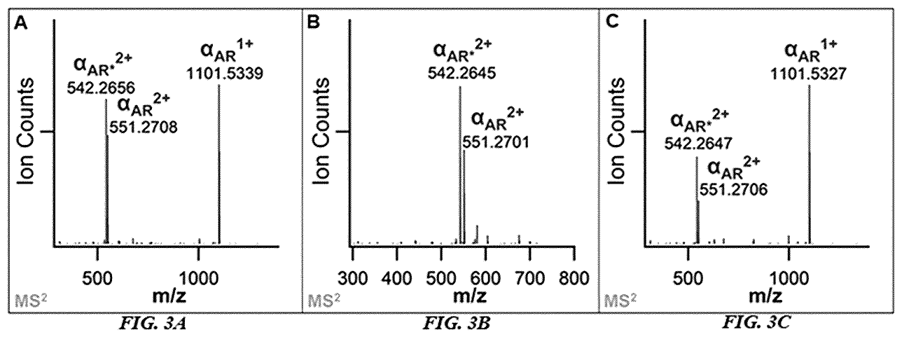

Characterization of TSTO Cross-linked Synthetic Peptide by MSⁿ Analysis TSTO cross-linking was first characterized on the synthetic peptide Ac-SR8 (Ac-SAKAYEHR) [SEQ ID NO:1]. Under the experimental conditions, three Ac-SR8 cross-linked products were detected: dead-end modified Ac-SR8 (α_{DN}), inter-linked Ac-SR8 homodimer [α-α], and Ac-SR8 homotrimer [α, α, α]. MS² analysis of dead-end modified Ac-SR8 (m/z 667.3163²⁺) yielded two dominant ions (m/z 542.2656²⁺, 551.2708²⁺) (see FIG. 2A). MS³ peptide fragment analysis determined these ions to be AR-modified Ac-SR8, with the lower mass ion corresponding to an AR moiety undergoing water loss (namely AR*), resulting in the detection of an ion doublet (α_{AR} and α_{AR*}) with mass difference (Δ) of 18.02 Da (see FIG. 2B-C). Two differently-charged species of inter-linked Ac-SR8 homodimer [α-α] were detected (m/z 773.7071³⁺, 580.5319⁴⁺), each fragmenting into dominant ions corresponding to α_{AR} and α_{AR*} during MS² analysis (see FIG. 3A-B). Similarly, MS² analysis of the Ac-SR8 homotrimer ti-link [α, α, α] (m/z 826.6508⁴⁺) yielded three dominant ions (α_{AR*}²⁺/α_{AR}²⁺/α_{AR}¹⁺) (see FIG. 3C). While MS³ analyses of both α_{AR} and α_{AR*} resulted in their unambiguous identification (see FIG. 2B-C), selecting AR*-modified peptides for sequencing would be preferred due to the AR moiety's propensity for dehydration.

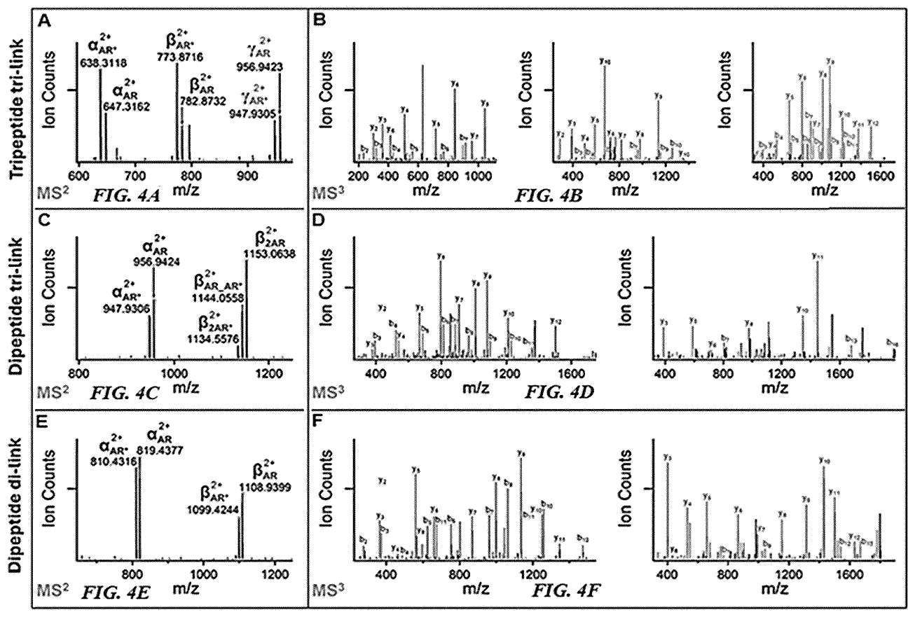

Characterization of TSTO Cross-linked BSA by MSⁿ Analysis. To characterize TSTO cross-linking in proteins, XL-MS analysis was performed on the model protein BSA, focusing on TSTO inter-linked peptides. As a result, all three types (I-III) of TSTO inter-linked peptides were identified by LC MSⁿ, each displaying the characteristic MS² fragmentation as expected. This is illustrated by MSⁿ analyses of representative TSTO cross-linked BSA peptides (see FIG. 4). For a tripeptide tri-link [α, β, γ] (m/z 795.7120⁶⁺), its MS² analysis yielded three sets of dominant ions corresponding to α_{AR}/α_{AR*}, β_{AR}/β_{AR*}, and γ_{AR}/γ_{AR*} fragments (see FIG. 4A). As shown, MS³ analyses of the three cross-link fragments α_{AR*} (m/z 638.3130²⁺), β_{AR*} (m/z 773.8743), and γ_{AR*} (m/z 947.9347) identified a tripeptide TSTO tri-link among BSA lysines K228, K374, and K498 (see FIG. 4B). MS² analysis of a dipeptide tri-link [α-β₂] (m/z 1054.7530⁴⁺) resulted in two sets of dominant ion species: α_{AR}/α_{AR*}, and β_{2AR}/β_{AR_AR*}/β_{2AR*} (see FIG. 4C). The detection of a fragment triplet with 18 Da increments indicates that peptide R carries two modified lysines, whereas peptide a only contains a single modified lysine. MS³ analyses of α_{AR*} and β_{2AR*} identified their sequences as ³⁷²LAK_{AR*}EYEATLEECCAK³⁸⁶ [SEQ ID NO:4] and ⁴⁹⁰TPVSEK_{AR*}VTK_{AR*}CCTESLVNR⁵⁰⁷ [SEQ ID NO:5], respectively (See FIG. 3D), signifying a dipeptide tri-link [BSA:K374-BSA:K495, K498]. Finally, for a dipeptide bi-link [α-β] (m/z 992.7017⁴⁺), MS² fragmentation produced two dominant ion pairs: α_{AR}/α_{AR*}, and β_{AR}/β_{AR*} (see FIG. 4E); MS³ analyses of α_{AR*} and β_{AR*} determined a cross-link between BSA:K117 and BSA:K489 (see FIG. 4F).

In total, 823 redundant cross-linked spectra matches (CSMs) were identified, corresponding to 167 unique ones. Of these, 21 were tripeptide tri-links, 24 were dipeptide tri-links, and 122 were dipeptide bi-links. Overall, tri-links contributed ˜27% (45/167) of the total unique CSMs. Breaking down tripeptide and dipeptide tri-links into their respective constituent residue pairs, a combined total of 118 K—K pairs were identified, with 37 being contributed by both TSTO tri- and bi-links, whereas 50 and 31 were unique contributed by TSTO bi-links and tri-links, respectively.

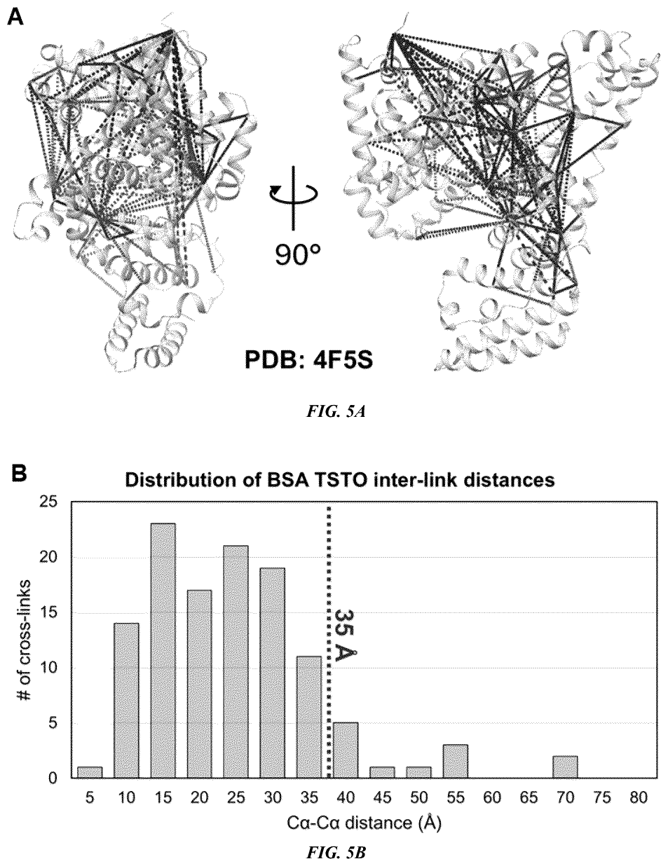

Considering the spacer arm length of TSTO (˜14 Å), lysine residues with Cα-Cα distance ≤35 Å were expected to be preferentially cross-linked. When mapped to the high-resolution crystal structure of BSA (PDB:4F5S) (see FIG. 5A), the overall mapped distance median was 21.1 Å with a satisfaction rate of cross-links under ≤35 Å of 90%. Taken together, these results demonstrate that TSTO is effective for protein cross-linking and the resulting cross-linked peptides exhibit unique MS² fragmentation patterns that are both predictable and reliable for unambiguous identification by LC MSⁿ analysis.

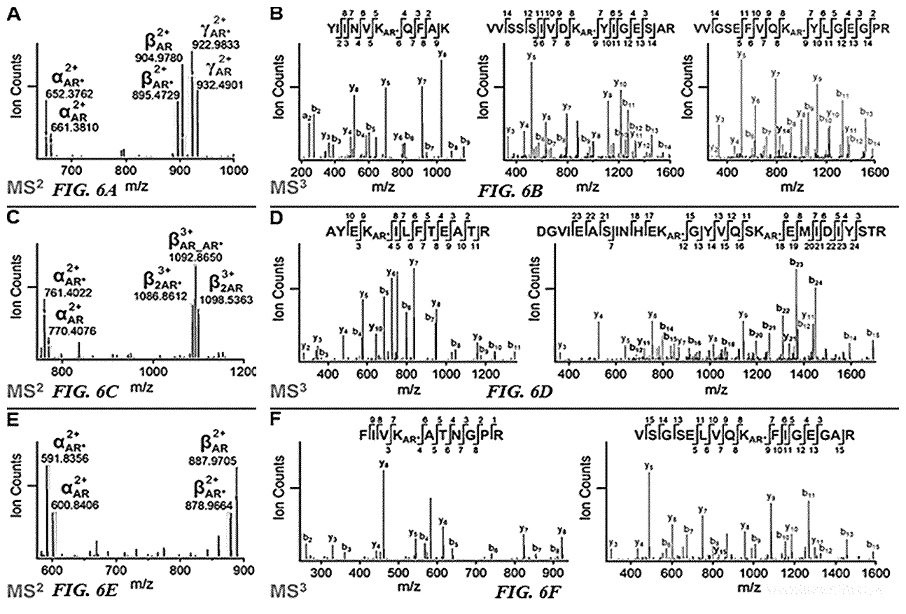

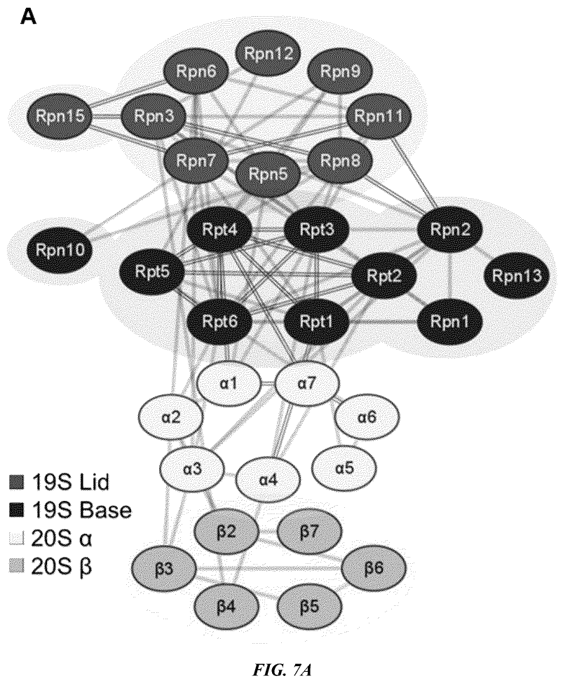

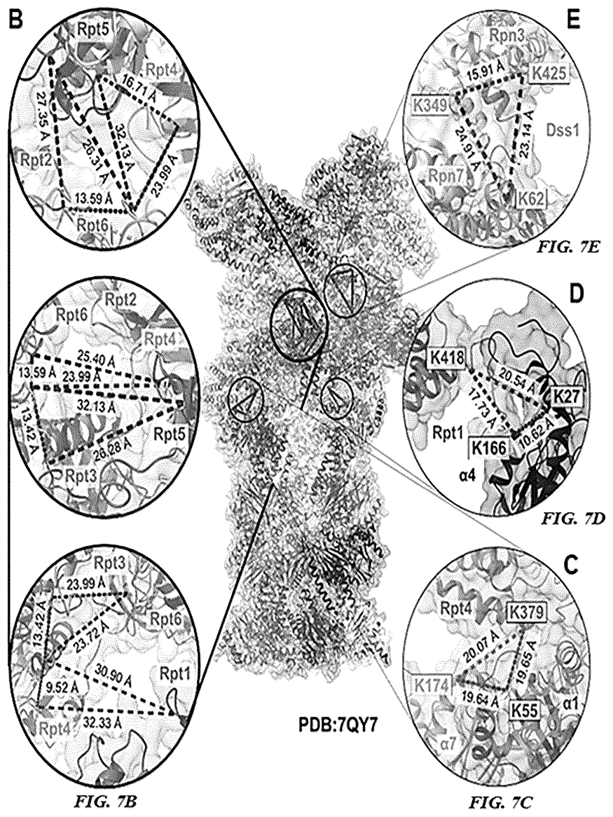

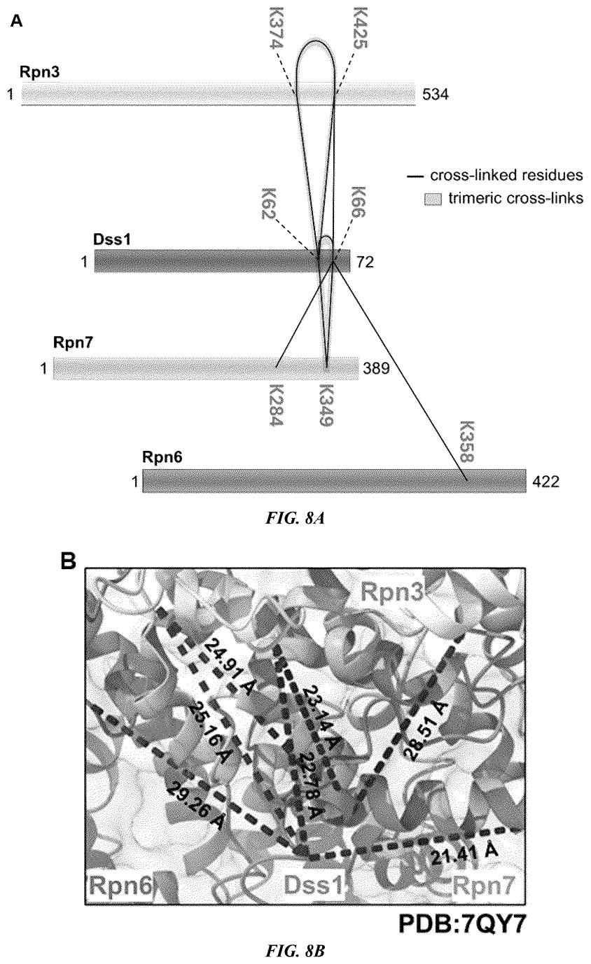

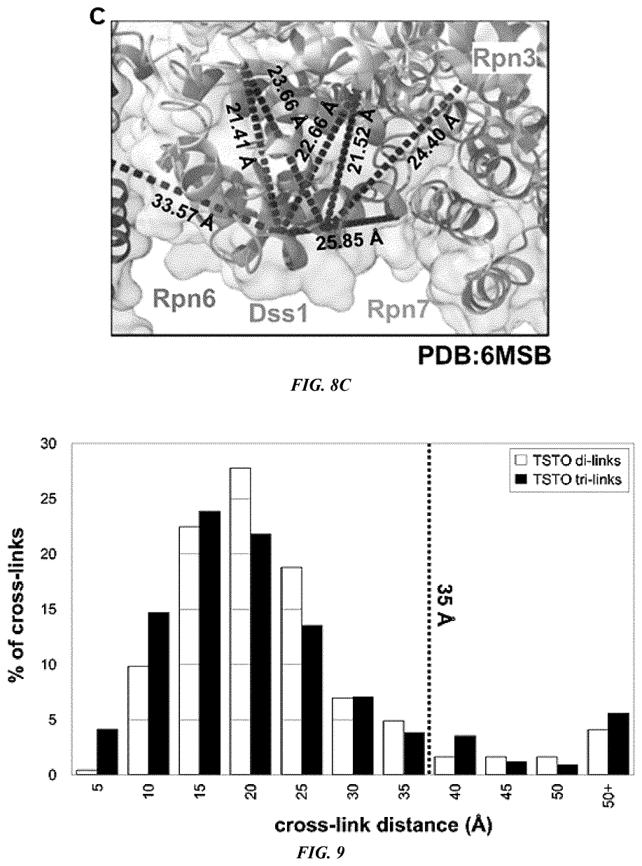

TSTO XL-MS Analysis of the 26S Proteasome. To explore TSTO's capability in XL-MS analysis of protein complexes, TSTO cross-linking of affinity-purified human 26S proteasomes was performed. Similar to BSA, all three types of TSTO inter-linked peptides of the 26S proteasome were detected (see FIG. 6). This was exemplified by MSⁿ analyses of a representative tripeptide ti-link [α, β, γ] (m/z 998.9354⁵⁺) among Rpt1:K116, Rpt4:K206, and Rpt3:K238 (see FIG. 6A-B), a dipeptide ti-link [α-β₂] (m/z 1209.0961⁴⁺) [Rpn12:K281-Rpn3:K455, K461](see FIG. 6C-D), and a dipeptide bi-link [α-β] (m/z 773.4180⁴⁺) between Rpt4:K72 and Rpt6:K222 (see FIG. 6E-F). Cumulatively, TSTO-based XL-MS analysis of the 26S proteasome resulted in a total of 808 unique CSMs. Of these, 41.3% were tri-links, indicating that TSTO can efficiently cross-link three spatially proximal residues within the proteasome. To assess the accuracy of TSTO cross-links, they were mapped to a high-resolution 26S proteasome structure (PDB: 7QY7). As a result, all of the mapped tri-links and bi-links have ˜90% satisfaction rates (≤35 Å), supporting their validity (see FIG. 5). To understand whether the introduction of a supplementary reactive group is beneficial for describing protein topologies, a TSTO XL-PPI map was constructed of the 26S proteasome comprising 92 edges (see FIG. 7A). Compared to previous XL-MS studies using bifunctional linkers, 31 additional inter-subunit PPIs were identified—with most of them at interaction interfaces between proteasomal subcomplexes. To better understand the benefits of TSTO, the interactions captured by its tri-links were examined. Among the 35 trimeric interactions within the proteasome, 13 described multimeric interactions among the six ATPase subunits (Rpt1-6), illustrating their proximity within the 19S base subcomplex (see FIG. 7B). In addition, the connectivity between the 19S and 20S subcomplexes was described by three trimeric interactions, including one among Rpt4, α1 and α7, and one between Rpt1 and α4 (see FIG. 7C-D). Apart from trimeric interactions involving three proteins, TSTO was able to concurrently place two distant residues of one protein (α4: K27, K166) to another (Rpt1:K418), due to their proximity in the three-dimensional structure. Another tri-link involving Rpt6 and α3 exemplifies the capability of TSTO to capture interactions involving adjacent residues (Rpt6:K397, K402) that may be missed by bifunctional linkers due to the tendency to form intra-links (loop-links). Moreover, TSTO cross-links placed a small proteasome subunit Dss1 in proximity to Rpn3, Rpn6, and Rpn7 within the 19S lid (see FIG. 7E, and FIG. 8), which has not been reported by previous XL-MS studies of the human 26S proteasome. Nonetheless, these interactions were supported by the two human 26S structures (PDB: 6MSB and 7QY7) as all cross-links between Dss1 and Rpn3, Rpn6, and Rpn7 have Cα-Cα distances ≤26 Å, falling below the distance threshold (see FIG. 7E, and FIG. 9). Collectively, these results have demonstrated that TSTO is effective in capturing multimeric interactions of proteasome complexes, providing three-dimensional contacts for the first time to support the spatial organization of these proximal subunits.

Integrative Structure Modeling Using Synthetic and Experimental XL-MS Data. To determine the value of trivalent versus bivalent cross-links, integrative structure modeling was applied to compute the structure of the proteasome base subcomplex (subunits Rpt1-Rpt6 and Rpn2). First, synthetic datasets were generated for the proteasome base subcomplex, replicating the experimental distance distribution of the proteasome TSTO dataset to systematically compare trifunctional and bifunctional cross-linkers. Subsets of 20, 30, 40, 60, and 120 cross-links were randomly sampled based on the distance probability distribution, with at least five replicates created for each subset size. For this analysis, a cross-linking site was defined as the set of residues bridged by a single cross-linker. To illustrate the uniqueness of trifunctional linkages, the analysis was focused on the comparison of trivalent and bivalent cross-links for integrative modeling. For each trivalent cross-link, a corresponding bivalent cross-link utilizing two of the three cross-linked lysines was used to ensure that the synthetic data reflects the spatial restraints of each cross-linker, providing a robust foundation for structural modeling.

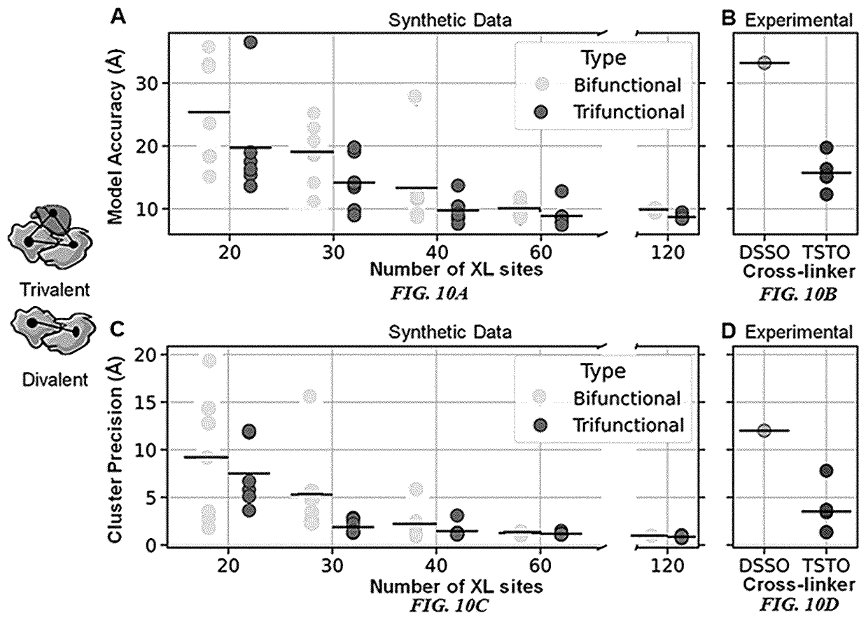

Ensembles of the proteasome subcomplex configurations that satisfy the input information (i.e., the model) were found by exhaustive Monte Carlo sampling guided by the scoring function, starting with random initial configurations of the rigid components. The models computed using trivalent cross-links were generally more accurate and precise than the ensembles computed using bivalent ones. The accuracy is defined as the average Cu root-mean-square deviation (RMSD) between the cryo-EM structure (PDB ID: 5GJR) and each of the structures in the ensemble, while the precision is defined as the average RMSD between all solutions in the ensemble. Increasing the number of both trivalent and bivalent cross-links used for modeling improved model accuracy and precision, with diminishing returns observed as the number of cross-links increased from 40 to 120. For datasets containing bivalent cross-links, the average model accuracy plateaued at ˜9.8 Å, while for trivalent cross-links the plateau occurred at ˜8.7 Å. The trivalent cross-linking data consistently produced better model accuracies, with average RMSDs of 19.7, 14.1, 9.7, 8.8, and 8.7 Å for datasets with 20, 30, 40, 60, and 120 cross-links, respectively. In contrast, the bivalent cross-linking data resulted in higher average RMSDs of 25.3, 19.0, 13.3, 9.7, and 9.8 Å for the same subset sizes. A similar trend was observed for the cluster precisions (see FIG. 10). Furthermore, the structure of the base subcomplex of the 26S human proteasome was computed using integrative modeling with experimentally derived DSSO and TSTO cross-linking datasets. The DSSO dataset included 83 cross-links within the base subcomplex. The TSTO dataset comprised 18 trivalent and 143 bivalent cross-links; from the TSTO data, 65 bivalent cross-links were randomly selected (five replicates) to ensure that the total number of cross-linking sites was consistent between the DSSO and TSTO datasets. Models computed using the TSTO dataset were considerably more accurate than models computed with the DSSO dataset (15.3 Å vs 33.2 Å). These results highlight TSTO's ability to provide richer structural information, such as the spatial positioning of three protein regions, which is critical for generating accurate structural models.

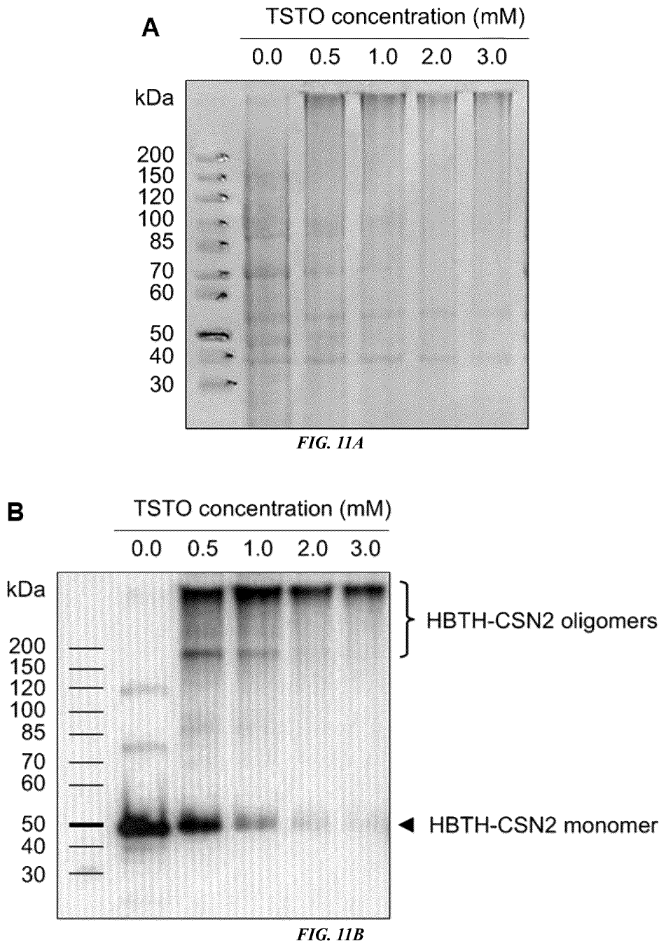

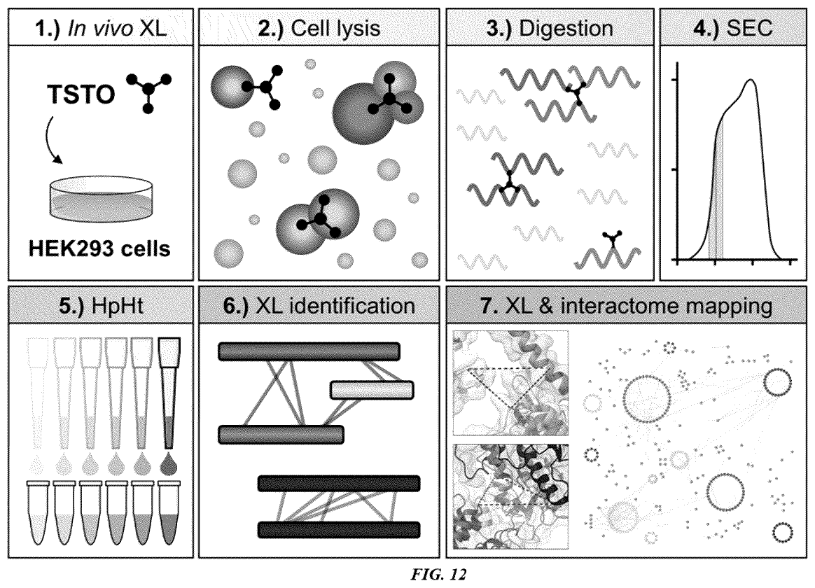

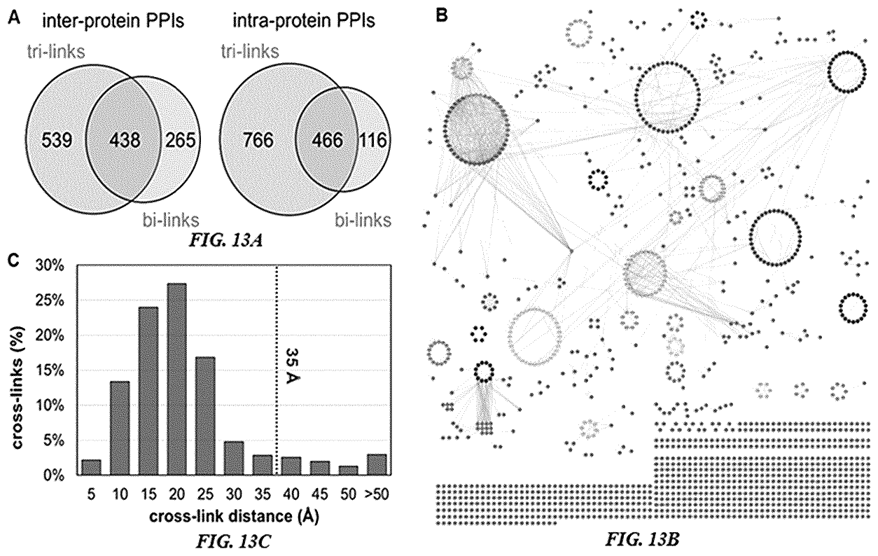

In Vivo TSTO Cross-linking of the HEK293 Cells. Next was investigated whether TSTO was applicable for system-wide XL-MS analysis to delineate cellular networks. To this end, we performed TSTO cross-linking of HEK293 cells stably expressing HBTH-tagged CSN2, a subunit of the COP9 signalosome. To evaluate TSTO in-cell cross-linking efficiency, immunoblot analysis was carried out to probe the formation of CSN2-containing cross-linked products, which were represented by high molecular weight protein bands. Based on the formation of CSN2-containing oligomer and its increased abundance with increasing cross-linker concentration, TSTO was determined to be membrane-permeable and suited for in-cell cross-linking (see FIG. 11). The general TSTO-based in vivo XL-MS workflow is illustrated in FIG. 12, in which cross-linked peptides were subjected to two-dimensional peptide separation prior to LC-MSⁿ analysis. Across two biological replicates, we identified a total of 9079 unique CSMs, of which 32.3% were tri-links. As shown, TSTO tri-links increased the total PPI yield by ˜27% compared to bi-link data alone (see FIG. 13A). Altogether, TSTO in-cell cross-linking yielded an XL-proteome of 1512 proteins containing 1242 PPIs (see FIG. 13B). These results indicate that TSTO cross-linking of intact cells is effective, and the presence of ti-links remains abundant in increasingly complex systems. Importantly, accurate identification of TSTO cross-links at the systems level was achieved using LC-MSⁿ.

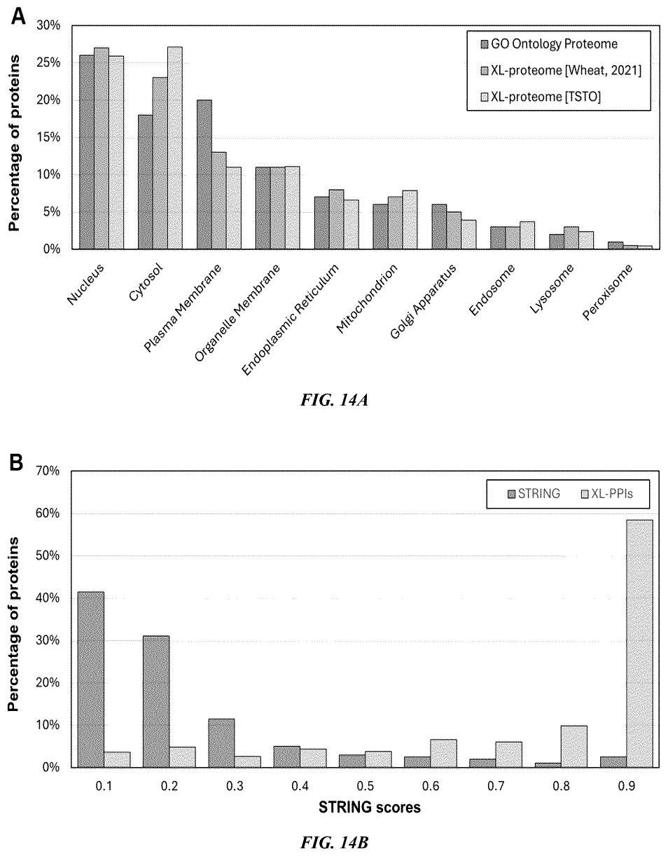

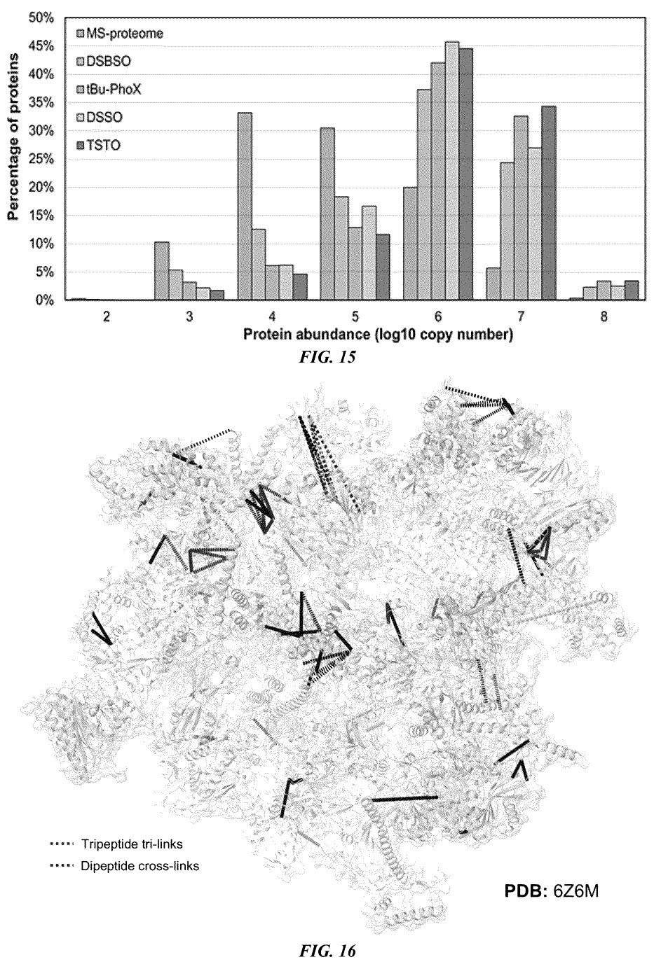

To examine the validity of the TSTO cross-links, they were first mapped to available high-resolution structures of protein complexes identified here (see FIG. 13C). In total, 1790 K—K linkages were mapped across 539 CORUM complexes and 95% of them were considered satisfactory (≤35 Å). Next, a gene ontology (GO) analysis was performed, which confirmed that the TSTO XL-proteome covers a wide range of molecular functions, biological processes, and cellular components (see FIG. 14A). Compared to BioGRID and BioPlex databases, 48% of the TSTO XL-PPIs were known and 52% were novel. The STRING scores were found for 501 of the XL-PPIs and ˜70% were determined to be above 0.8 (see FIG. 14B), indicating high-confidence interactions. Overall, among the 1242 inter-protein PPIs identified within this TSTO in vivo dataset, roughly one-third were novel when compared to an aggregate of recent systems-level cross-linking studies. This suggests that while most XL-PPIs are supported, TSTO provides information that was not detected by bifunctional MS-cleavable reagents. Finally, to estimate the dynamic range of the XL proteome captured by TSTO cross-linking, the abundance distribution of the identified cross-linked proteins based on their copy numbers as determined by shotgun proteomics was plotted (see FIG. 15). Compared to the MS-proteome, the TSTO XL-proteome was shifted towards higher abundance proteins, however, it is comparable to previous proteome-wide XL-MS studies. Nevertheless, TSTO is capable of targeting cellular proteins across all cellular compartments and capturing interactions among proteins spanning five orders of magnitude. Collectively, these results demonstrate that TSTO is effective for global profiling of endogenous PPIs from their cellular environments.

Multimeric Interactions of In Vivo Protein Complexes. Importantly, TSTO XL-MS has enabled the identification of multimeric interactions within various protein complexes. One well-represented complex is the 80S ribosome machinery. Specifically, extensive interactions describing subunit proximities were identified, particularly those between the 40S or 60S subcomplexes. This allows for an in-depth description of ribosomal PPIs with 3-D contacts (see FIG. 16). Interestingly, trimeric interactions were also found between 80S and several putative ribosome-binding partners, providing structural insights underlying their functional relevance in protein synthesis. Of these, the most frequent was SERBP1 (SERPINE mRNA-binding protein 1), which was shown to interact with 60S ribosome subunits RPL7A, RPL27, and RPL34 through several tri-links. While eukaryotic SERBP1 (Stm1 in yeast) has been associated with dormant ribosomes due to its role in clamping 40S and 60S subunits together to prevent mRNA access, it has been shown to primarily contact 40S subunits. Interestingly, TSTO identified ribosome-SERBPT cross-links spanning the 80S, including distant 60S subunits. In current high-resolution structures of ribosome-bound SERBP1, the majority of SERBP1 is unresolved-likely buried within the 80S. Together, the results suggest that the interaction of SERBP1 with ribosomal proteins is extensive, contacting various 40S and 60S subunits to inactivate 80S machinery. Another trivalent cross-link was identified between 40S ribosome subunits and UBAP2L (Ubiquitin-associated protein 2-like). While UBAP2L is a known RNA-binding protein (RBP) that may associate with ribosomes to facilitate protein synthesis, its function and roles remain poorly characterized. TSTO identified a trimeric interaction between UBAP2L and neighboring 40S subunits RPS7 and RPS27, triangulating its position near the small ribosomal complex and correlating well with the role of the 40S in initial binding and reading of mRNA.

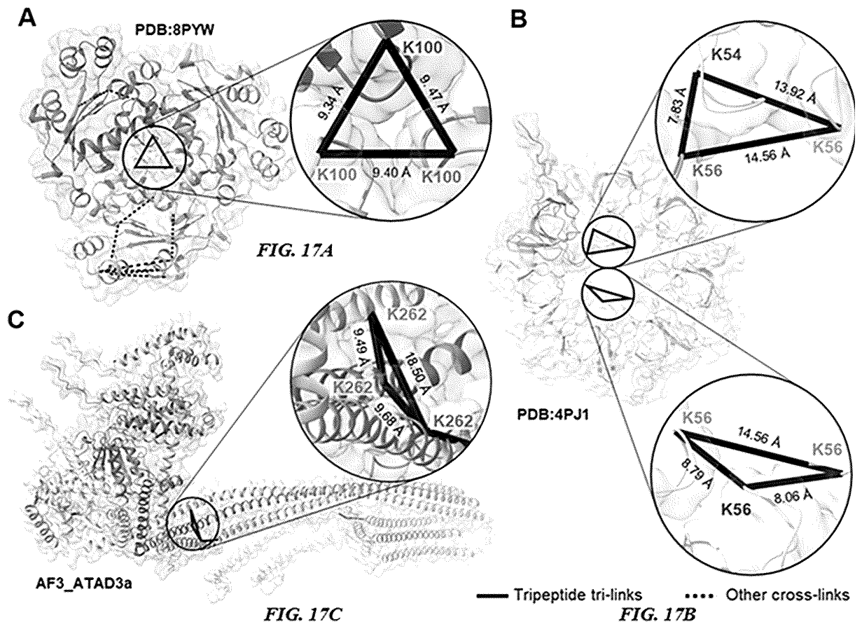

One notable aspect is TSTO's unique ability to define trimers, especially homomeric trimers. This has been challenging for bifunctional linkers due to the difficulty in identifying multimeric interactions. For instance, the trimer of nucleoside-disphosphate kinase B (NME2) was detected due to the identification of a TSTO tri-link connecting three identical sequences containing NME2:K100. Interestingly, NME2 is known to form homohexameric structures comprising two stacked homotrimers. When mapped to a high-resolution structure of NME2 (PDB:8PYW), the loop regions containing the K100 residues of each homotrimer were found to localize along the axis of the hexamer, within 9.5 Å of one another (see FIG. 17A). Similarly, a homotri-link was identified for the 10 kDa mitochondrial heat shock protein (HSPE1) through its K54 and K56 residues, suggesting an oligomeric complex. Indeed, HSPE1 has been shown to form a homoheptameric ring within the human mitochondrial chaperonin ‘football’ complex (PDB: 4PJ1), in which the longest distance spanned between any pair of HSPE:K54 or HSPE:K56 residues within neighboring triplets was 20 Å (see FIG. 17B). In addition, homotrimeric TSTO cross-links were identified from proteins that are known to assemble into oligomeric complexes but currently lack high-resolution structures. For instance, ATPase family AAA domain-containing protein 3A (ATAD3A) was shown to oligomerize through a specific residue (K262). Using AlphaFold3, we predicted the structure of an ATAD3A trimer at high confidence (90>plDDT>70) and mapped all possible K262 interactions satisfactorily (<18.5 Å) (see FIG. 17C), exemplifying the capability of TSTO to facilitate structural modeling of protein oligomers.

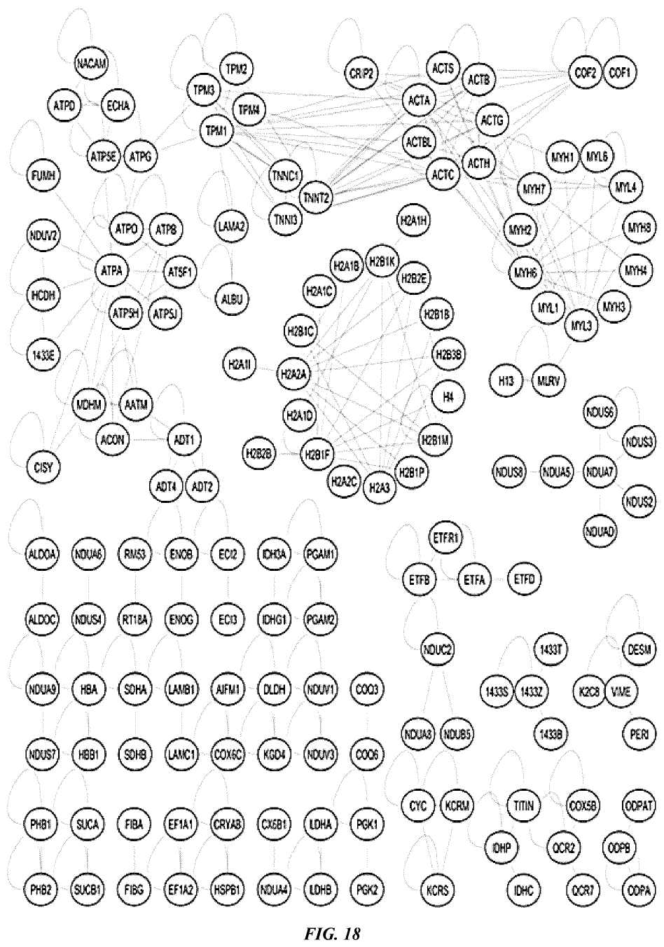

In Vivo TSTO Cross-linking of Mouse Heart Tissue. To illustrate the feasibility of TSTO in another biological context, in-tissue cross-linking was performed to identify cross-linked peptides using the same workflow for in-cell XL-MS as established above. Here, TSTO cross-linking of intact mouse hearts resulted in the identification of a total of 4,770 CSMs using LC MSⁿ. Among these, 170 unique trivalent and 921 unique bivalent cross-links were identified, providing a snapshot of protein interactions within the heart. The most abundant XL-PPIs involve proteins that are major structural and functional components of cardiac tissue (see FIG. 18), including actin, myosin, tropomyosin, and ATP synthase subunits. This observation correlates with the known role of these proteins in heart muscle function, particularly in the sarcomere—the contractile unit of muscle fibers.

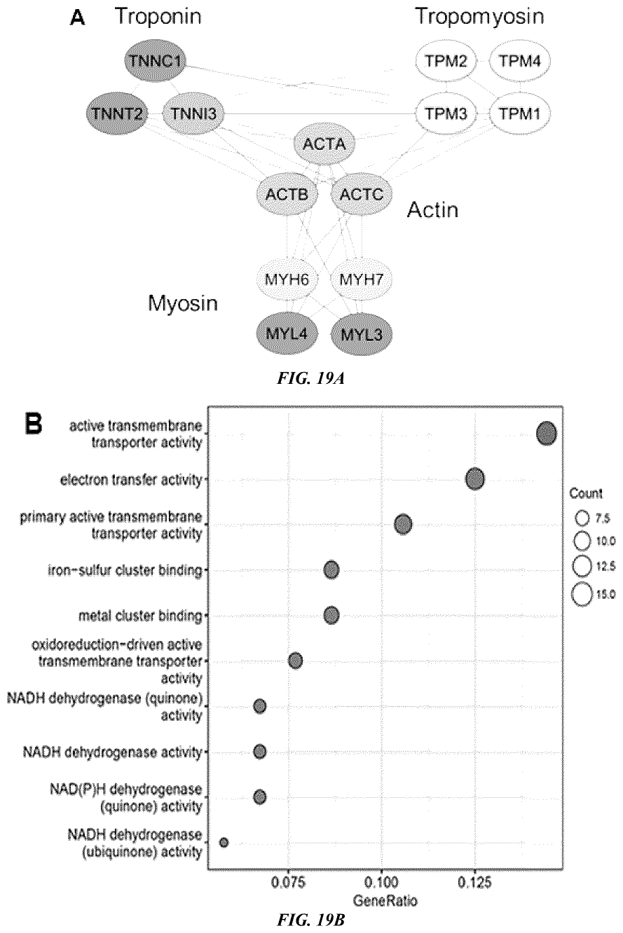

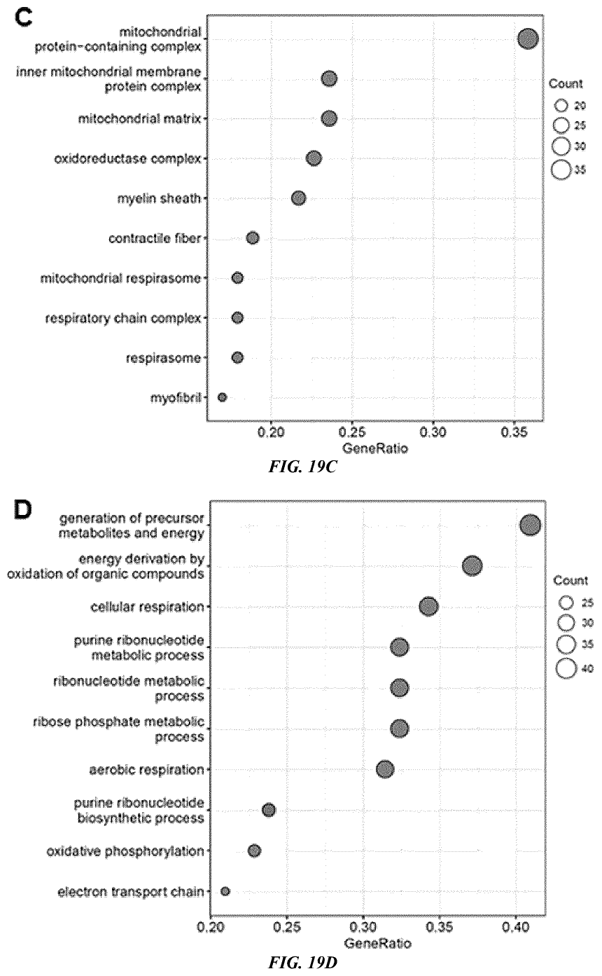

To further explore protein interactions within the cardiac muscle, an XL-PPI map was generated, focusing on the interactions of cardiac actins with myosins, tropomyosins, and troponins, all of which are key regulators of muscle contraction (see FIG. 19A). Myosin, a motor protein, binds actin and its function is regulated by tropomyosin and troponin. Tropomyosin, a coiled-coil protein, runs along actin filaments and controls myosin access to actin-binding sites, while the troponin complex (composed of troponin C, T, and I) binds actin and tropomyosin to modulate contraction by controlling myosin-binding site exposure. Consistent with these molecular functions, our cross-linking data revealed interactions among these proteins, with the most direct connections observed between myosin and actin, while tropomyosin and troponins were cross-linked with both actin and each other (see FIG. 19A). Notably, trivalent cross-links were identified not only within myosins and tropomyosins but also between troponin and actin, as well as between actin and tropomyosin. Additionally, it was found that tropomyosin was cross-linked to an ATP synthase subunit. While this interaction is not typically associated with sarcomere function, it could suggest a potential regulatory link between contractile and energy-producing processes, possibly reflecting coordinated control of ATP synthesis and muscle contraction in cardiac cells. Further Gene Ontology (GO) enrichment analysis of heart-specific proteins captured by TSTO cross-linking revealed a significant representation of pathways associated with cellular energy metabolism, particularly those involved in ATP synthesis and mitochondrial function, which are critical for maintaining cardiac contractility and overall heart function (see FIG. 19B-D). Taken together, the results have shown the feasibility of TSTO for in-tissue cross-linking, expanding its applicability for different biological samples.

Summarizing the results using TSTO for cross-linking mass spectrometry. In summary, the disclosure provides for the design, synthesis, and characterization of a novel trioxane-based, MS-cleavable, membrane-permeable homotrifuctional cross-linker, TSTO. This cross-linker enables simultaneous cross-linking of up to three proteins, allowing for more in-depth PPI analysis to complement existing reagents. As demonstrated in the studies herein, all types of TSTO cross-linked peptides display unique and predictable CID-induced fragmentation, which can be unambiguously identified using an LC MSⁿ analysis workflow. TSTO's unique capability to concurrently cleave all three cross-linker arms makes it a brand-new class of MS-cleavable reagent that yields a lower number of theoretical MS fragment ions per peptide constituent than current MS-cleavable reagents, simplifying ion selection for subsequent MS³ analysis. While MS²-based data acquisition has become popular for analyzing conventional MS-cleavable cross-linked peptides. It is envisaged that MS³-based approaches will be preferred for the identification of tripeptide cross-links as co-fragmentation of three peptides within a single spectrum would heavily convolute database searching, impeding identification of higher-order cross-linked species by MS²-based approaches.

Moreover, the trioxane presents a novel core structure for developing multifunctional MS-cleavable cross-linkers as one arm can be replaced in lieu of other functional groups—such as enrichment or reporter tags—to enable cross-link purification or further improve cross-link detection and identification. As with TSTO, the cleavage of the trioxane within the mass spectrometer would release any additional functional groups, preventing their impact on cross-linked peptide identification. It is noted that NHS ester groups in TSTO can be replaced by other reactive chemistries to target specific or non-specific amino acids. Thus, the development of TSTO presented here opens a new direction for designing diverse cross-linkers to further advance XL-MS technologies. Furthermore, the disclosure demonstrates the successfully deployment of the TSTO-based XL-MS platform to capture trimeric and dimeric interactions of protein complexes and cellular networks from intact cells, offering an alternative avenue for expanding PPI mapping at the proteome level. The new molecular details on endogenous protein complexes can be coupled with AlphaFold prediction and/or integrative structural modeling to better elucidate the structural organization of cellular networks in future studies. Therefore, the TSTO-based XL-MS platform represents a highly promising approach for advancing XL-MS technology towards systems structural biology in vivo.

A number of embodiments have been described herein. Nevertheless, it will be understood that various modifications may be made without departing from the spirit and scope of this disclosure. Accordingly, other embodiments are within the scope of the following claims.

1. A trioxane-based mass spectrometry (MS)-cleavable cross-linker comprising:
a central trioxane group, which may be isotopically enriched with heavier isotopes selected from C¹³, and O¹⁸;
two or more MS-cleavable bonds;
two or more reactive cross-linking groups that can react with amino acids of peptides and/or proteins;
optionally, linker arms that connect the reactive cross-linking groups with the central trioxane group, wherein the linker arms may be isotopically enriched with heavier isotopes selected from H², C¹³, O¹⁸ and N¹⁵;
wherein the trioxane-based MS-cleavable cross-linker is configured to form a dimeric or trimeric cross-links with amino acids of peptides and/or proteins.

2. The trioxane-based MS-cleavable cross-linker of claim 1, wherein the two or more MS-cleavable bonds can be cleaved using collision-induced dissociation.

3. The trioxane-based MS-cleavable cross-linker of claim 1, wherein the two or more reactive cross-linking groups are located equal distant to the central trioxane group.

4. The trioxane-based MS-cleavable cross-linker of claim 1, wherein the two or more reactive cross-linking groups are selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative.

5. A trioxane-based mass spectrometry (MS)-cleavable cross-linker, wherein the trioxane-based MS-cleavable cross-linker has the structure of: 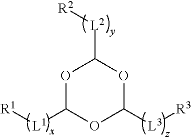
wherein,
L¹, L², and L³ are linker arms each individually selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide, 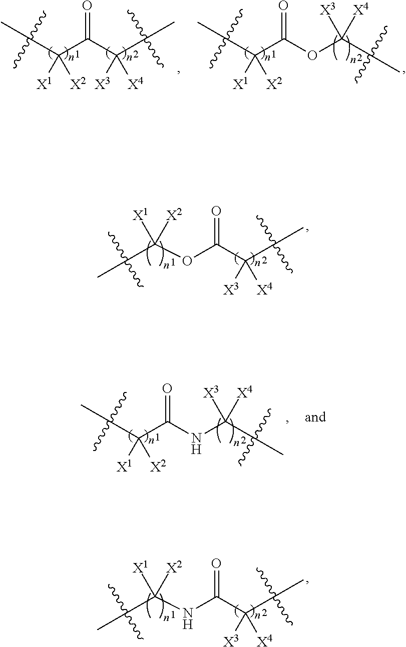
wherein L¹, L², and L³ may be isotopically enriched with heavier isotopes selected from H², C¹³, O¹⁸ and N¹⁵;
R¹, R², and R³ are individually selected from a reactive cross-linking group that can react with amino acids of peptides and/or proteins, an enrichable handle, and a fluorophore, wherein at least two of R¹, R² and R³ are reactive cross-linking groups;
X¹-X⁴ are each individually selected from H, (C₁-C₆)alkyl, (C₁-C₆)alkenyl, (C₁-C₆)alkynyl, cyano, azide, hydroxyl, aldehyde, carboxyl, halo, amide, and amine, wherein each of the foregoing groups may be isotopically enriched with heavier isotopes selected from H², C³, O¹⁸ and/or N¹⁵;
x, y, and z are integers selected from 0 and 1; and
n¹ and n² are integers selected from 0, 1, 2, 3, 4, 5 and 6,
wherein the trioxane-based MS-cleavable cross-linker is configured to form dimeric or trimeric cross-links with amino acids of peptides and/or proteins.

6. The trioxane-based MS-cleavable cross-linker of claim 5, wherein L¹, L², and L³ are individually selected from optionally substituted (C₁-C₆)alkyl, 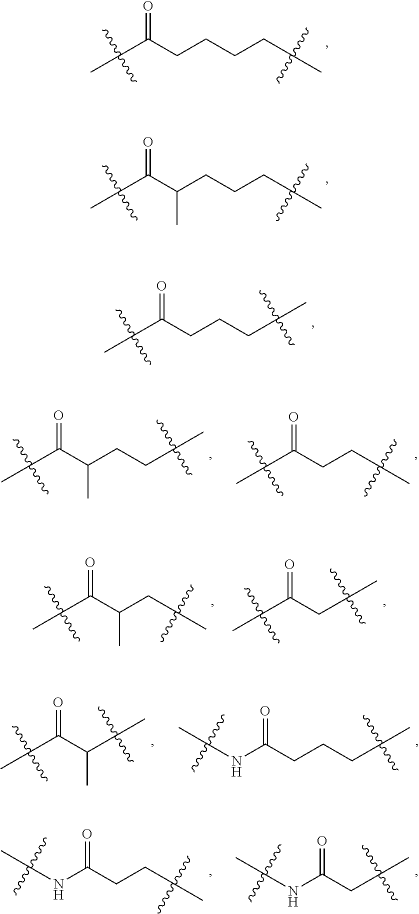
wherein each of the foregoing groups may be isotopically enriched with heavier isotopes selected from H², C¹³, O¹⁸ and N¹⁵.

7. The trioxane-based MS-cleavable cross-linker of claim 6, wherein L, L², and L³ are

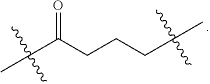 [SMILES: *CCCC(*)=O]

8. The trioxane-based MS-cleavable cross-linker of claim 5, wherein the reactive cross-linking group is selected from an optionally substituted N-hydroxysuccinimide (NHS) ester, an optionally substituted hydrazide, an optionally substituted maleimide, a haloacetamide, a sulfosuccinimidyl suberate, an optionally substituted aldehyde, an optionally substituted diazirine, an optionally substituted azido-methyl-coumarin, an optionally substituted benzophenone, an optionally substituted anthraquinone, and an optionally substituted psoralen derivative.

9. The trioxane-based MS-cleavable cross-linker of claim 5, wherein the reactive cross-linking group that can react with amino acids of a peptide or protein are selected from

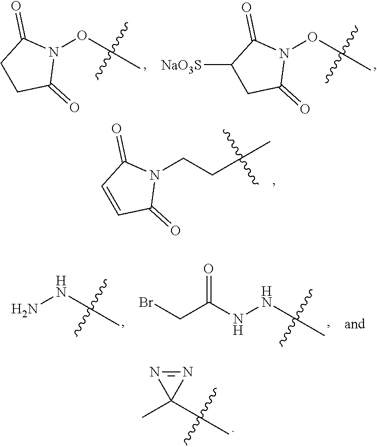

10. The trioxane-based MS-cleavable cross-linker of claim 5, wherein the enrichable handle is selected from a click chemistry linker, a phosphate, a phosphonate, a fluorophore, biotin, an azide, an alkyne, and a phosphonic acid.

11. The trioxane-based MS-cleavable cross-linker of claim 5, wherein R¹, R², and R³ are reactive cross-linking groups, and wherein:
(i) two of R¹, R², and R³ have same structure, or
(ii) R¹, R², and R³ have same structure, or
(iii) R¹, R², and R³ have different structures.

12. The trioxane-based MS-cleavable cross-linker of claim 5, wherein the MS-cleavable trioxane-based cross-linker has the structure of: 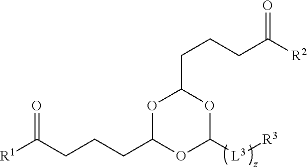
wherein,
L³ is a linker arm selected from an optionally substituted (C₁-C₁₀)alkyl, an optionally substituted (C₁-C₁₀)alkenyl, an optionally substituted (C₁-C₁₀)alkynyl, a (C₁-C₈)alkoxy, an ester, an amide, 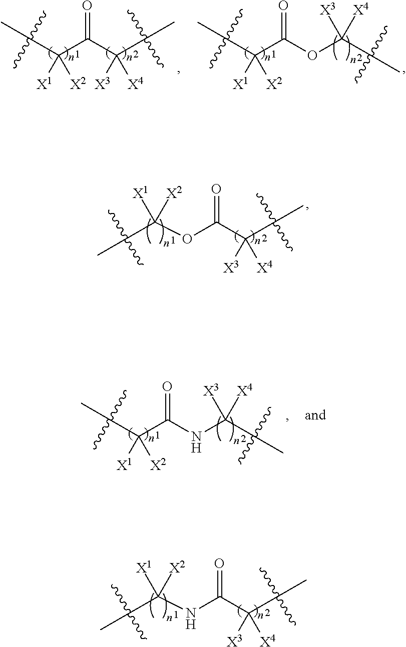
wherein L³ may be isotopically enriched with heavier isotopes selected from H², C¹³, O¹⁸ and N¹⁵;
R¹ and R² are 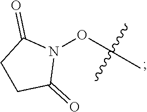 [SMILES: *ON1C(=O)CCC1=O]
R³ is selected from an enrichable handle, a fluorophore, 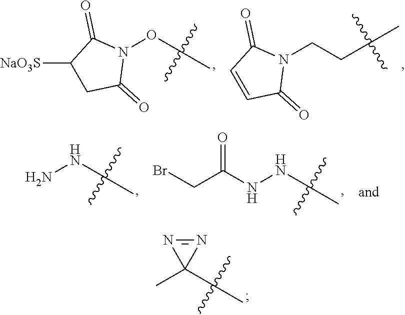
z is an integer selected from 0 and 1; and
n¹ and n² are integers selected from 0, 1, 2, 3, 4, 5 and 6.

13. The trioxane-based MS-cleavable cross-linker of claim 5, wherein the MS-cleavable trioxane-based cross-linker has the structure of: 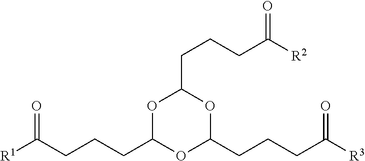
wherein,
R¹-R³ are each individually selected from

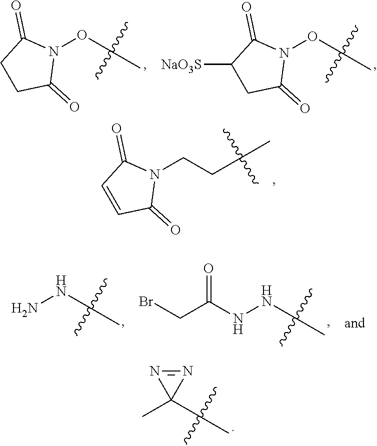

14. The trioxane-based MS-cleavable cross-linker of claim 5, wherein the MS-cleavable trioxane-based cross-linker is tris-succinimidyl trioxane (TSTO) having the structure of:

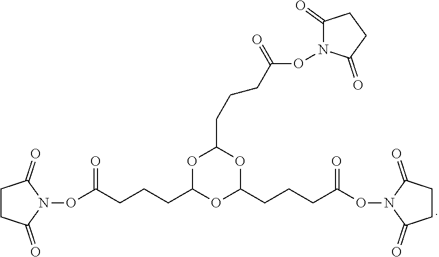 [SMILES: O=C(CCCC1OC(CCCC(=O)ON2C(=O)CCC2=O)OC(CCCC(=O)ON2C(=O)CCC2=O)O1)ON1C(=O)CCC1=O]

15. A method for mapping intra-protein interactions in a protein, inter-protein interactions in a protein complex, or any combination thereof, the method comprising:
contacting the protein and/or the protein complex with the trioxane-based MS-cleavable cross-linker of claim 1 to form a cross-linked product;
digesting the cross-linked product to form a plurality of fragments, wherein a portion of the plurality of fragments comprises cross-linked peptide fragments; and
identifying and analyzing cross-linked peptide fragments using tandem mass spectrometry (MSⁿ) to map intra-protein interactions in the protein and/or inter-protein interactions in the protein complex.

16. The method of claim 15, wherein each trioxane-based MS-cleavable cross-linker interacts with 2 to 3 lysine residues of the protein and/or protein complex.

17. The method of claim 15, wherein a data-dependent MS³ acquisition method is used for identifying and analyzing the cross-linked peptide fragments.

18. A method for mapping global protein-protein interactions (PPIs) from a sample comprising a plurality of proteins;
contacting the sample comprising a plurality of proteins the trioxane-based MS-cleavable cross-linker of claim 1 to form a cross-linked product to form crosslinked proteins;
digesting the crosslinked proteins to form crosslinked protein fragments or peptides;
isolating fractions that are enriched with cross-linked protein fragments or peptides in the sample;
analyzing the fractions using tandem mass spectrometry (MSⁿ) and protein database searching to identify cross-linked protein fragments or peptides; and
mapping the identified cross-linked protein fragments or peptides to generate a global structural map of PPIs.

19. The method of claim 18, wherein the sample is a tissue sample or a cellular sample.

20. The method of claim 18, wherein each trioxane-based MS-cleavable cross-linker interacts with 2 to 3 lysine residues of the plurality of proteins.


SEQUENCE LISTING (14 sequences)

SEQ ID NO: 1 - protein, 8 aa, synthetic construct
SAKAYEHR

SEQ ID NO: 2 - protein, 12 aa, synthetic construct
CASIQKARFGER

SEQ ID NO: 3 - protein, 14 aa, synthetic construct
VTKARCCTESLVNR

SEQ ID NO: 4 - protein, 17 aa, synthetic construct
LAKAREYEATLEECCAK

SEQ ID NO: 5 - protein, 22 aa, synthetic construct
TPVSEKARVTKARCCTESLVNR

SEQ ID NO: 6 - protein, 14 aa, synthetic construct
LCVHEKARTPVSEK

SEQ ID NO: 7 - protein, 19 aa, synthetic construct
ETYGDMADCCEKARQEPER

SEQ ID NO: 8 - protein, 12 aa, synthetic construct
YIINVKARQFAK

SEQ ID NO: 9 - protein, 18 aa, synthetic construct
VVSSSIVDKARYIGESAR

SEQ ID NO: 10 - protein, 18 aa, synthetic construct
VVGSEFVQKARYLGEGPR

SEQ ID NO: 11 - protein, 14 aa, synthetic construct
AYEKARILFTEATR

SEQ ID NO: 12 - protein, 31 aa, synthetic construct
DGVIEASINHEKARGYVQSKAREMIDIYSTR

SEQ ID NO: 13 - protein, 12 aa, synthetic construct
FIVKARATNGPR

SEQ ID NO: 14 - protein, 18 aa, synthetic construct
VSGSELVQKARFIGEGAR